(19) United States
(12) Patent Application Publication
(10) Pub. No.: US 2006/0281090 A1
(43) Pub. Date: Dec. 14, 2006

(54) Capsid-modified adenovirus vectors and methods of using the same

(75) Inventors: Andre Lieber, Seattle, WA (US); Dmitry M. Shayakhmetov, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: UNIVERSITY OF WASHINGTON UW TECH TRANSFER- INVENTION LICENSING, SEATTLE, WA (US)

(21) Appl. No.: 10/559,752

(22) PCT Filed: May 3, 2004
(86) PCT No.: PCT/US04/13717

§ 371 Date: Dec. 6, 2005

Related U.S. Application Data
(60) Provisional application No. 60/466,858, filed on May 1, 2003.

(51) Int. Cl. C12Q 1/68; C12P 21/06

(52) U.S. Cl. 435/6; 435/69.1

(57) ABSTRACT

Adenovirus fiber mutated in the regions involved in the recognition and the binding of blood factor proteins, and adenoviruses comprising such fibers are provided.

### CROSS-REFERENCES TO RELATED APPLICATIONS

This application claims the benefit of U.S. Provisional Application No.60/466,858, filed May 1, 2003, the disclosure of which is incorporated by reference herein in its entirety.

### STATEMENT AS TO RIGHTS TO INVENTIONS MADE UNDER FEDERALLY SPONSORED RESEARCH OR DEVELOPMENT

This work was supported by NIH grants P01 HL53750, R01 CA 80192, P30 DK 47754 and HL-00-008. The Government may have certain rights in the invention.

### BACKGROUND OF THE INVENTION

Adenoviruses (also referred to herein as “Ads”) are used in an increasing number of applications for gene transfer. Adenoviruses have been identified in numerous animal species. They exhibit low pathogenicity, are nonintegrative and replicate both in dividing and quiescent cells. Adenoviruses generally exhibit a broad host spectrum and are capable of infecting a very large number of cell types, such as epithelial cells, endothelial cells, myocytes, hepatocytes, nerve cells and synoviocytes.

The adenoviral genome is a double-stranded linear DNA molecule of about 36 kilobases containing genes encoding the viral proteins. At the ends of the adenoviral genome, inverted repeats (also referred to as inverted terminal repeats or ITRs) contain replication and the encapsidation regions. The early genes are distributed in four regions dispersed in the adenoviral genome (designated E1 to E4). The early genes are expressed in six transcriptional units. The late genes (designated L1 to L5) partly overlap with the early transcription units and are generally transcribed from the major late promoter (referred to as MLP).

Recombinant adenoviral vectors are derived from adenoviruses and usually include cis acting regions that are necessary for the replication of the virus in the infected cell (e.g., the ITRs and encapsidation sequences). Recombinant adenoviral vectors also contain substantial internal deletions designed to remove viral genes, to allow for the insertion of a heterologous gene(s) for gene transfer. To accommodate heterologous genes, adenoviruses used in gene transfer protocols are usually deficient for replication by deletion of at least the E1 region and are propagated in a complementation host cell line that provides in trans the deleted viral function(s). One commonly used host cell line, the 293 line, was established from human embryonic kidney cells and provides the adenoviral E1 function in trans (see, e.g., Graham et al., J. Gen. Virol. 36:59-72 (1977)).

The adenoviral infectious cycle occurs in two steps. The early phase precedes the initiation of replication and makes it possible to produce the early proteins regulating the replication and transcription of the viral DNA. The replication of the genome is followed by the late phase during which the structural proteins that constitute the viral particles are synthesized. The assembly of the new virions takes place in the host cell nucleus. In a first stage, the viral proteins assemble so as to form empty capsids of icosahedral structure into which the genome is encapsidated. The assembled virus includes a penton base and fiber. The adenoviruses liberated are capable of infecting other permissive cells. The fiber and the penton base present at the surface of the capsids play a role in the cellular attachment of the virions and their internalization. During infection, the adenovirus binds to a cellular receptor present at the surface of the permissive cells via a trimeric adenoviral fiber. The virus particle is then internalized by endocytosis through the binding of the penton base to cellular integrins (e.g., αᵥβ₃ and αᵥβ₅). In this regard, soluble adenoviral fiber or anti-fiber antibodies can inhibit infection by the adenovirus.

The adenoviral trimeric fiber is composed of 3 domains: (1) A tail is located at the N-terminal (proximal) end of the fiber. The tail is highly conserved from one serotype to another. The tail interacts with the penton base and ensures the anchorage of the molecule in the capsid. (2) A shaft (also referred to as a stem) is connected to the tail. The shaft is in the form of a rod and composed of a number of repeats of β sheets of amino acids. The number of β sheet repeats varies according to the serotype. (3) At the C-terminal (distal) end of the shaft, a globular fiber knob is present that contains the trimerization signals. The fiber knob also can include a binding site(s ) for a native cellular receptor(s).

The specificity of infection of an adenovirus is determined by a binding site(s) on the fiber(s) for a native cellular receptor, which can be situated at the surface of permissive cells. The binding site and the cellular receptor, can be different depending on the serotype of the adenovirus. Native cellular receptors can include, for example, the class I major histocompatibility complex and fibronectin as primary receptor and as cofactor, respectively, for adenoviruses. However, other proteins also can be involved. For example, the cellular receptor for the coxsackie viruses (also referred to as the coxsackievirus-adenovirus receptor or CAR) is recognized by type 2 and 5 adenoviruses. CD46 is the native cellular receptor for adenovirus type 35.

In vivo, the interaction of adenoviruses with permissive cells is more involved. Adenoviruses can infect a single tissue, or a variety of tissues, depending on the native cellular receptor. For example, the CAR receptor, recognized by Ad5, is expressed on liver cells and a variety of other cells types. In vitro, Ad5 vectors use a two-step mechanism to infect cells. The first step is a high-affinity interaction between the Ad fiber knob and CAR. This interaction involves a flexible, long (22-β-repeat) fiber shaft. Attachment to CAR is followed by binding of viral penton RGD motifs to cellular integrins, triggering virus internalization. In contrast, in vivo the interaction of Ad5 with CAR and integrins is unlikely to be the major pathway for adenoviral infection of liver cells. Several studies have documented that mutations that abolish CAR and integrin interactions are not sufficient to eliminate liver transduction.

Most studies on the interactions of adenovirus vectors with the host upon systemic application have been done with human Ad5 virus. Following systemic administration, Ad5 viruses are cleared from the blood stream and accumulate in the liver. Clearance of Ad5 from the bloodstream and its accumulation in the liver begins to occur within minutes. This phenomenon is thought to be due to the tissue architecture and vascular systems of the liver. Intravenously injected adenovirus particles reach the liver through the portal vein and contact most hepatocytes only after passing through the liver sinusoids, the walls of which are formed by endothelial cells. Sinusoid endothelial cells have fenestrae of about 100 nm. These fenestrae of the sinusoidal lumen allow communication with the space of Disse, which is in direct contact with hepatocytes because of the lack of a continuous basement membrane. Kupffer cells are located on the inside of the sinusoidal wall. The fenestration within the sinusoidal wall allows Ad5 particles (which have an average diameter of 80 nm) to efficiently translocate from the plasma to hepatocytes. Both hepatocytes and Kupffer cells efficiently take up Ad5 particles.

Systemic application of adenoviruses is associated with toxicity. The initiation of virus-associated toxicity does not depend on adenovirus gene expression, but is mediated by initial virus interactions with host cells. Systemic adenovirus administration can be associated with the production and release of cytokines (such as interleukin 6 (IL-6), IL-10, IL-8, tumor necrosis factor alpha (TNF-α), and gamma interferon (IFN-γ)) and chemokines (such as MIP-1α and MIP-2) within hours after the intravenous injection of Ad5 vectors. These cytokines, in turn, play a major causative role in the pathologic changes (e.g., vascular leakage, liver damage, and so forth) associated with intravenous adenovirus infusion. In addition, cytokine production is believed to play a major causative role in the induction of an antiviral immune response.

The rapid clearance of adenovirus upon systemic administration, as well as the toxic effects associated with adenovirus administration, are impediments to adenovirus mediated gene transfer. Currently the mechanisms responsible for Ad clearance and innate toxicity are poorly understood. Thus, there is a need for a further understanding of these mechanisms and a need for adenoviruses and adenoviral vectors that exhibit reduced clearance and/or toxicity in vivo. The present invention satisfies this and other needs.

### BRIEF SUMMARY OF THE INVENTION

The present invention provides capsid-modified adenoviruses having adenovirus fiber mutated in the regions involved in the recognition and the binding of blood factor proteins. Also provided are adenoviruses comprising such fibers, infected host cells comprising such adenoviruses, and methods of preparing infectious adenovirus particles.

In one aspect, a mutant adenovirus fiber is provided. The fiber includes a binding site for a cellular receptor and a mutation of a residue in or affecting a blood factor protein binding site. The mutation is characterized in that the residue is directed to the binding site for a blood factor protein. The mutation reduces the affinity or avidity of the fiber for the blood factor protein. The blood factor protein binding site can be, for example, Factor IX, TFPI, C3-precursor, complement C4, complement C4BP, hemopexin, fibrinogen, elastase-1, pregnancy zone protein, or the like.

In certain embodiments, the adenovirus fiber comprises a mutation in the fiber knob or shaft. In some embodiments, the mutation can be in the adenovirus fiber knob. For example, the mutation can be in the AB, FG, and/or HI loops. In other embodiments, the mutation can be in the AB, BC, CD, DE, EF, FG, GH, HI, and/or IJ β loop(s). In additional embodiments, the mutation can be in the A, B, C, D, E, F, G, H, I and/or J sheet(s). In yet other embodiments, the adenovirus fiber comprises a short shaft.

In a typical embodiment, the binding affinity of the fiber for its native cellular receptor is not substantially reduced. In an exemplary embodiment, the adenovirus fiber comprises a mutation in a loop in the fiber knob, wherein the affinity of the fiber knob for the native cellular receptor of the adenovirus fiber is not substantially reduced. Such an adenovirus fiber can be, or can be derived from, for example, an Ad2, Ad5 or Ad35 fiber. In exemplary embodiments, the adenovirus fiber knob can be derived from an Ad2, Ad5 or Ad35 fiber knob. Such an adenovirus fiber knob can comprise, for example, a mutation of a residue in the AB, EF and/or HI exposed loop(s). In a specific embodiment, the binding affinity of the fiber for the blood factor protein can be ablated.

The adenovirus fiber optionally can comprise a ligand capable of recognizing a cell surface molecule different from the native cellular receptor for adenovirus fiber. The ligand can be, for example, an antibody, a peptide, a lipid, a glycolipid, a sugar, or the like. The ligand can be inserted at any suitable location in the adenovirus fiber, such as, for example, at the C-terminal end of the fiber, in the HI loop, in capsid protein IX, in the penton, in the hexon, or the like.

In another aspect, a DNA fragment or expression vector encoding the adenovirus fiber is provided. Such a fiber typically includes a binding site for a cellular receptor and a mutation of a residue in or affecting a blood factor protein binding site. The mutation reduces the affinity or avidity of the fiber for the blood factor protein. The DNA fragment can optionally include a fiber further encoding a ligand.

In yet another aspect, a cell line comprising the DNA fragment is provided. The DNA fragment can be, for example, integrated into the genome or in the form of an episome in the cell. The DNA is typically operatively linked to a promoter for expression of the adenovirus fiber in the cell line. The cell line optionally can further include a function encoded by the E1, E2, E4 and/or L1-L5 region and capable of complementing an adenovirus deficient in a function encoded by the E1, E2, E4 and/or L1-L5 region. For example, the cell line can be, for example, the 293 cell line or a derivative thereof.

A further aspect of the invention provides an adenovirus comprising the mutant adenovirus fiber. Such a fiber typically includes a binding site for a cellular receptor and a mutation of a residue in or affecting a blood factor protein binding site. The mutation reduces the affinity or avidity of the fiber for the blood factor protein. The DNA fragment can optionally include a fiber further encoding a ligand. In certain embodiments, the adenovirus lacks a functional native fiber and comprises mutant adenovirus fiber. The adenovirus fiber can optionally include a ligand capable of recognizing a cell surface molecule different from the native cellular receptor for the adenovirus. The ligand can be, for example, an antibody, a peptide, a lipid, a polypeptide, a glycolipid, a sugar or the like. The ligand can be inserted at any suitable location in the adenovirus, such as in the fiber. In an exemplary embodiment, the ligand is inserted at the C-terminal end of the fiber, in the HI loop, in capsid protein IX, in the penton, or in the hexon.

The adenovirus can be a replication-competent, a replication-defective or a replication-attenuated recombinant adenovirus. The adenovirus can be deleted, for example, for all or part of the E1 region and, optionally, for all or part of the E3 region. In some embodiments, the adenovirus can be deleted for all or part of the E2, E4 and/or L1-L5 region.

In another aspect, the adenovirus is a recombinant adenovirus comprising a gene of interest and a mutant adenovirus fiber. Such a fiber typically includes a binding site for a cellular receptor and a mutation of a residue in or affecting a blood factor protein binding site. The mutation reduces the affinity or avidity of the fiber for the blood factor protein. The DNA fragment can optionally include a fiber further encoding a ligand. In certain embodiments, the adenovirus lacks a functional native fiber and comprises mutant adenovirus fiber. The adenovirus fiber can optionally include a ligand capable of recognizing a cell surface molecule different from the native cellular receptor for the adenovirus. The ligand can be, for example, an antibody, a peptide, a lipid, a polypeptide, a glycolipid, a sugar or the like. The ligand can be inserted at any suitable location in the adenovirus, such as in the fiber. In an exemplary embodiment, the ligand is inserted at the C-terminal end of the fiber, in the HI loop, in capsid protein IX, in the penton, or in the hexon. The gene of interest can, for example, encode a cytokine, a cellular receptor, a nuclear receptor, a ligand, a blood coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection, an antibody, a toxin, an immunotoxin, a marker or the like.

In a further aspect, a method of producing an adenovirus comprising the mutant adenovirus fiber. Such a fiber typically includes a binding site for a cellular receptor and a mutation of a residue in or affecting a blood factor protein binding site. The mutation reduces the affinity or avidity of the fiber for the blood factor protein. The method generally includes transfecting a genome encoding the adenovirus into a host cell line, culturing the transfected host cell line under appropriate conditions to allow the production of the adenovirus, and optionally recovering the adenovirus from the culture of the transfected host cell line. The method can optionally further include substantially purifying the adenovirus.

In yet a further aspect, a host cell infected with an adenovirus comprising the mutant adenovirus fiber is provided. Such a fiber typically includes a binding site for a cellular receptor and a mutation of a residue in or affecting a blood factor protein binding site. The mutation reduces the affinity or avidity of the fiber for the blood factor protein.

In another aspect, a pharmaceutical composition is provided comprising an adenovirus including the mutant adenovirus fiber. Such a fiber typically includes a binding site for a cellular receptor and a mutation of a residue in or affecting a blood factor protein binding site. The mutation reduces the affinity or avidity of the fiber for the blood factor protein. The adenovirus can be combined with a pharmaceutically acceptable carrier. Also provided is a pharmaceutical composition comprising an infected host cell in combination with a pharmaceutically acceptable carrier. The infected host cell comprises an adenovirus including the mutant adenovirus fiber. The adenovirus or infected host cell can be used therapeutically or prophylactically for the preparation of a medicament intended for the treatment of the human or animal body by gene therapy.

In yet another aspect, a method for increasing the efficacy of adenovirus administration is provided. The method generally includes administering to a subject a recombinant adenovirus comprising a gene of interest and a mutant adenovirus fiber. The mutant adenovirus fiber comprises a binding site for a cellular receptor and a mutated blood factor protein binding site, wherein the affinity of the fiber for the blood factor protein is substantially reduced. The cellular receptor can be, for example, a native cellular receptor. The binding site for the cellular receptor can be, for example, a ligand. The ligand can be, for example, an antibody, a peptide, a hormone, a polypeptide or a sugar.

The gene of interest can be, for example, a cytokine, a cellular receptor, a nuclear receptor, a ligand, a coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection, an antibody, a toxin, an immunotoxin, a marker, or the like. The gene of interest optionally can be operatively linked to a promoter. The promoter can be, for example, a tissue-specific promoter.

In yet another aspect, a method for reducing toxicity associated with adenovirus administration is provided. The method generally includes administering to a subject a recombinant adenovirus or adenoviral vector comprising a gene of interest and a mutant adenoviral fiber comprising a binding site for a cellular receptor and a mutated blood factor protein binding site, wherein the affinity of the fiber for the blood factor protein is substantially reduced. The cellular receptor can be, for example, a native cellular receptor or a ligand. The ligand can be, for example, an antibody, a peptide, a hormone, a polypeptide, a sugar, or the like. The gene of interest can be, for example, a cytokine, a cellular receptor, a nuclear receptor, a ligand, a coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection, an antibody, a toxin, an immunotoxin, a marker, or the like.

### BRIEF DESCRIPTION OF THE DRAWINGS

FIG. 1. Analysis of the role of fibers in liver Ad tropism. (a) Infectivity of AdFF/6His (native fiber deleted) and Ad5 (fiber intact) on 293 and 293-DH26 cells (expressing both the artificial receptor for His tag and CAR). n=3. (b) Analysis of vector DNA concentration in the livers of C57B1/6 mice. Ad5 or AdFF/6His (both expressing luciferase as a reporter gene) were injected into the tail vein. Control mice (c) were injected with saline. To ensure equivalent DNA loads, the membrane was hybridized with a mouse β-glucuronidase specific probe (lower panel). (c) Luciferase activity measured in liver lysates 3 days after injection of Ad5L and AdFF/6His. (d) Analysis of vector persistence in the blood. Viruses were injected into the portal vein of C57Bl/6 mice. Three and 15 min after injection, blood samples were collected from the vena cava inferior, obtained plasma was serially diluted and used for infection of 293-DH26 cells. Luciferase activity in infected cells was analyzed 24 h post infection. n=4.

FIG. 2. Analysis of hepatocellular transduction in wild-type mice, Ldlr^{-/-}, and Ldlr^{-/-}/ApoE^{-/-} knockout mice. Pre-injection of LDL reduced infection with Ad5L1/2, Ad5F*, and Ad5/35 vectors 4, 10, and 15 fold, respectively. To test whether the LDLR could serve as a receptor for the Ad blood factor complexes, the in vivo infectivity of the non-CAR binding Ad5F* and Ad5/35L vectors in wild type and in Ldlr^{-/-} mice was compared. The absence of LDLR expression in hepatocytes did not affect transduction with these vectors to the extent of the competition with lactoferrin in wild-type mice. Injection of Ldlr^{-/-} mice with lactoferrin reduced the efficiency of gene transfer to a similar degree as seen in wild-type mice. This result indicates that lactoferrin interacting receptors provide a major contribution to Ad liver transduction. The observed reduction of Ad infectivity in Ldlr^{-/-} mice could be due to elevated blood LDL levels which interfere with function of the lactoferrin receptors LRP and HSPG.

FIG. 3. The role of blood factors in Ad transduction of hepatocytes in vivo and analysis of potential receptors mediating the uptake of complexes between Ad and blood factors. (a and b) Ad5L1 and Ad5F* express GFP and luciferase as reporters. Ad5L2 and Ad5/35L express β-galactosidase (β-Gal) as a reporter. In settings “with blood”, Ad5L1/2, Ad5/35L or AD5F* were injected into the portal vein through a permanently placed catheter. Fifteen minutes later, hepatocytes were isolated for culture and analysis of reporter gene expression. In settings “without blood”, the portal vein and v. cava inferior were canulated and blood was flushed from the liver through the portal vein. Virus was infused through the portal vein and the circulation between v. porta and v. cava was closed allowing asanguinous isolated liver perfusion with virus containing saline. Thirty minutes after virus application, hepatocytes were isolated by collagenase perfusion. In both settings, reporter gene expression in plated hepatocytes was analyzed 48 hours post infection. n=4 for Ad5L1, Ad5F*, Ad5L2 and Ad5/35L variants with blood, and for Ad5L1 without blood. n=5 for Ad5L2 variant without blood, and n=7 and 8 for Ad5F* and Ad5/35L variants without blood, respectively. ** P<0.001. There were no statistically significant differences between other paired variants. (c) Hepatocyte transduction with GFP-expressing Ad viruses (UV-fluorescence). (d) Hepatocyte transduction with β-galactosidase expressing Ad viruses after histochemical staining with X-gal. (e) Competition of Ad infection with different ligands to hepatocellular receptors. The indicated competitors or saline (as a control) were injected into the portal vein through a catheter 5 minutes before virus administration. Hepatocytes were purified and reporter gene activities were analyzed as described in Example 1. n=3 for Control, pBSA, ASF and hLDL groups, and n=5 for lactoferrin group. * P=0.0056 for hLDL Ad5L1/2 group, compared to control. For the other viruses in the hLDL group and all viruses in the lactoferrin group there was a significant difference in hepatocytes transduction compared to the Control group (P<0.001). (f) Heparinase I was administrated into the tail vein of C57B1/6 mice 30 min before virus injection. Fifteen minutes after virus application, hepatocytes were harvested, plated, and analyzed for reporter gene activities two days post infection. n=3 for all Control groups, n=4 for Ad5L1/2, n=5 for Ad5F* and Ad5/35L (heparinase treated) groups. * P<0.01; ** P<0.0001.

FIG. 4. Interaction of adenovirus fiber knob domain with coagulation factor IX and HSPG mediates CAR-independent infection of mouse hepatocytes in vitro. (a) Isolated asanginous liver perfusion of C57B1/6 mice with virus or saline plus human FVIII or FIX (3 U/ml). After virus perfusion, hepatocytes were isolated and transgene expression was analyzed. n=4 for each virus group. ** P<0.0001. (b) Dose-response relationship for FIX as a mediator for hepatocyte transduction in vivo. The perfusions were performed as described in (a) (1 unit per ml of recombinant human FIX corresponds to the physiological FIX concentration in human plasma) (n=4). (c) Surface plasmon resonance analysis of direct FIX-Ad fiber knob interaction. Only direct binding of Ad fiber knobs to immobilized FIX can generate an increase in the refractive index. Note that Ad5 knob more efficiently binds to FIX than the Ad35 knob. (TBS—Tris-buffered saline.) The SPR analysis was conducted in triplicates. Representative changes in the refractive index over time for each fiber knob domain are shown. (d) Competition of FIX-mediated Ad5F* infection on HepG2 cells with recombinant purified Ad5 and Ad35 fiber knob domains. Indicated amounts of fiber knobs (μg/ml) were incubated with 0.5 U of FIX for 15 minutes before addition of virus at an MOI of 1000 virus particles per cell. Following additional incubation for 15 minutes, the virus containing mixtures were added to cells and incubated for 2 hours. Luciferase activity in transduced cells was analyzed 48 hours post infection. n=4. *−P<0.01. (e) Infection of MEF Lrp^{+/+} or MEF Lrp^{-/-} cells by Ad5F* or Ad5/35L with or without FIX. (f) Transduction of HSPG-expressing (CHO-K1) and HSPG-negative (CHO-pgsA745) cells by Ad5F* or Ad5/35L with or without FIX. n=4. ** P<0.001.

FIG. 5. FIX-mediated infection of primary human hepatocytes and reduced infection of mouse liver with mutated Ad vector in vivo. (a) Visualization of GFP expression in primary human hepatocytes transduced with Ad5F* virus in the presence of FIX and different competitors 48 hours post infection. (b) Infection of primary human hepatocytes with Ad5L or Ad5F* viruses (MOI of 1000 virus particles per cell) in saline (control settings) or saline plus FIX, human lactoferrin, or heparin was done in triplicates. Luciferase activity was analyzed 48 hours post infection. The increase in luciferase expression in the presence of FIX was statistically significant (* P=0.0174, ** P=0.0033). (c) SPR analysis of direct binding of recombinant Ad5, Ad35 and Ad5mut knob domains to FIX. From top to bottom, the graph depicts results for Ad5, Ad35, Ad5mut and TBS, respectively.

FIG. 6. (a) Protein cross-linking analysis of interaction between Ad knob and recombinant human FIX. Purified recombinant Ad5 or Ad35 knob domains at increasing concentrations were incubated with human FIX and cross-linked as described in Methods. The products of the cross-linking reactions were developed by Western blotting with anti-FIX rabbit polyclonal antibodies. The FIX-specific cross-linking product of expected size is indicated by an arrow. Note that Ad35 knob interacts with FIX less efficiently than Ad5 knob, and no FIX-specific cross-linking product was detected after incubation of FIX with BSA. The lanes labeled as “FIX” and “0” show recombinant FIX only before and after cross-lining. (b) Quantitative Southern blot analysis of purified Ad5F* and Ad5mut vector stocks. Virus DNA purified from 10 μl of vector stock was applied (in two-fold serial dilutions) on an agarose gel together with serial dilutions of standard DNA (linearized adenovirus genome containing plasmid). After hybridization with a ³²P-labeled Ad specific DNA probe, and quantitation of signals with a phosphorimager, the concentrations of the indicated viruses were calculated. The concentration for Ad5F* was 8.6×10¹² virus particles per ml, and for Ad5mut was 6.8×10¹² virus particles per ml. (c) Transduction of 293-DH26 cells with Ad5F* and Ad5mut vectors. n=3. For all further studies, an MOI of 1000 virus particles per cell was used. (d) Infection of MEF1 (Lrp+/+) and MEF2 (Lrp−/−) cells with Ad5F* and Ad5mut in the presence or absence of FIX (3 U/ml). n=3. (e) Infection of CHO-K1 (HSPG+/+) and CHO-A475 (HSPG−/−) cells with Ad5F* and Ad5mut in the presence or absence of FIX (3 U/ml). n=4.

FIG. 7. In vivo transduction of hepatocytes with capsid-modified Ads and Southern blot analysis of Ad DNAs in the liver at different times after systemic vector application. (A) At 72 hours after intravenous Ad injection, livers were recovered and serial sections of formalin-fixed tissues were prepared. To visualize GFP fluorescence, images of sections were taken under UV light. Representative fields are shown. Magnification, ×200. (B) At the indicated times after intravenous Ad injection, livers were recovered from mice and total DNA was purified as described in Example 2. Ten micrograms of total DNA digested with HindIII enzyme was loaded on agarose gels along with serial threefold dilutions of standard (Ad5 genomic) DNA of a known concentration (Std). After transfer to Hybond N+ membranes, filters were hybridized with a mouse β-glucuronidase-specific probe to confirm equivalent loads (GUS). Subsequently, the membranes were stripped and rehybridized with an Ad-specific probe (Ad). The right panels show the quantification of vector genomes by PhosphorImager analysis (n=3); error bars indicate standard deviations. Note that the absolute amount of Ad DNA significantly decreased over time (compare the intensity of the Ad bands to the intensity of the standard bands). The control was DNA purified from the livers of mice injected with PBS only.

FIG. 8. Short-shafted vectors induce lower levels of proinflammatory cytokine and chemokine gene transcription after systemic Ad administration. At the indicated times, total liver RNA was purified, and the mRNA levels for proinflammatory genes were analyzed by an RNase protection assay as described in Example 2. Upregulated IL-1α and MIP-2 gene mRNA levels are indicated by arrows. (MCP-1, monocyte chemoattractant protein; GAPDH, glyceraldehyde 3-phosphate dehydrogenase.)

FIG. 9. Levels in plasma of proinflammatory cytokines, chemokines, and ALT are lower for short-shafted Ads than for long-shafted Ads. At the indicated times, plasma samples from three individual mice per virus treatment group were collected and analyzed in duplicate for levels of proinflammatory cytokines and ALT as described in Example 2. Error bars indicate standard deviations. A single asterisk indicates a P value of 0.05; double asterisks indicate a P value of 0.01.

FIG. 10. Alignment of adenovirus fiber knob domains for selected serotypes. The amino acids involved in the formation of β-sheet structures are in boxes. The designation of each β-sheet structure (A-J) is indicated above each box. Conservative and homologous residues are indicated in the consensus sequence. Exposed loops are located between the β-sheet regions. In this figure, the Ad5 fiber knob sequence is SEQ ID NO: 1; the Ad2 fiber knob is SEQ ID NO:2; the Ad12 fiber knob is SEQ ID NO:3; the Ad31 fiber knob is SEQ ID NO:4; the Ad9 fiber knob is SEQ ID NO:5; the Ad35 fiber knob is SEQ ID NO:6; and the Ad11 fiber knob is SEQ ID NO:7.

### DETAILED DESCRIPTION OF THE INVENTION

The present invention provides capsid-modified adenoviruses having adenovirus fiber mutated in the regions involved in the recognition and the binding of blood factor proteins. Also provided are adenoviruses comprising such mutant fibers, host cells expressing the mutant fibers, infected host cells comprising such adenoviruses, and methods of preparing infectious adenovirus particles.

The interaction of adenoviruses with blood factor proteins and infection of cells via a blood factor pathway(s) has been discovered to be related to toxicity associated with systemic (e.g., intravenous) adenovirus administration. Mutation of the adenovirus fiber to reduce or ablate the affinity or avidity for blood factor proteins alters the tropism of the viruses. Such viruses can, for example, exhibit reduced toxicity upon administration. As used herein, the terms “tropism-modified” and “altered tropism” refer to adenoviruses and vectors whose native tropism has been altered in some way (e.g., partially modified, or fully ablated). For example adenoviruses can exhibit tropism for cells involved in the defense system for initiating host innate and inflammatory responses. Such tropism is mediated by blood factor binding to the adenovirus fiber. The target cells include cells of the defense system for initiating host innate and inflammatory responses, such as for example, dendritic cells, splenic macrophages, Kupffer cells (residential liver macrophages), alveolar macrophages, endothelial cells (e.g., sinusoidal endothelial cells in the liver), parenchymal cells (e.g., in the lung, liver or spleen), cells of the bone marrow and lymph nodes, and the like.

By mutating the blood factor protein binding site to reduce the affinity or avidity of the fiber for its cognate blood factor protein, an adenovirus comprising mutant adenovirus fiber according to the present invention exhibits an altered tropism toward cells of the host defense system, whereby the initiation of innate and inflammatory responses is reduced or eliminated. Further, in certain embodiments, the amounts of such tropism-modified adenoviruses used can be reduced, or spared.

The adenovirus fiber is modified by mutation of one or more residues of the fiber, characterized in that the residues are directed toward or involved in a blood factor protein binding site on the fiber. The mutation(s) reduces the affinity or avidity of the mutant fiber for the blood factor protein. In this context, the term “mutation” refers to a substitution, deletion, and/or insertion of one or more residues in the adenovirus fiber. The mutation of blood factor protein binding site can reduce the affinity or avidity of the fiber for the blood factor protein by a factor of about 10, of about 100, of about 1000, of about 10,000, or about 100,000, or of about 1,000,000, or more. In certain embodiments, the blood factor protein binding site is ablated, meaning that no biologically significant blood factor protein binding is retained. The blood factor protein can be, for example, Factor IX (FIX), Tissue Factor Pathway Inhibitor protein (TFPI), C3-precursor, complement C4, complement C4BP, hemopexin, fibrinogen, elastase-1, pregnancy zone protein, or other blood factor. In certain embodiments, the blood factor protein can be a non-protein blood factor.

The mutations in the adenovirus fiber can be in the shaft and/or fiber knob. In certain embodiments, the mutations are in an exposed loop region of the adenovirus fiber knob. The three-dimensional crystallographic structure of the Ad5 fiber knob has been determined (see, e.g., Xia et al., Structure 2:1259-70 (1994)). Each fiber knob monomer contains 8 anti-parallel β sheets designated A to D and G to J and 6 major loops of 8 to 55 residues. The minor sheets E and F are considered to be part of the DG loop between the D and G β sheets (see also Table 1) (the sequence of the Ad5 fiber is also depicted in U.S. Patent Publication No. 2003/0175243, where +1 represents the initiator Met residue).

TABLE 1

| | |
|---|---|
| β sheet | loop |

| nomenclature | residues | nomenclature | residues |
|---|---|---|---|
| A | 400 to 403 | AB | 404 to 418 |
| B | 419 to 428 | — | — |
| C | 431 to 440 | CD | 441 to 453 |
| D | 454 to 461 | DG | 462 to 514 |
| G | 515 to 521 | GH | 522 to 528 |
| H | 529 to 536 | HI | 537 to 549 |
| I | 550 to 557 | IJ | 558 to 572 |
| J | 573 to 578 | | |

β sheets A, B, C and J constitute the V sheets directed toward the viral particle. The other four β sheets D, G, H and I form the R sheets, which are involved in binding to the cellular receptor. The V sheets appear to play a role in the trimerization of the structure.

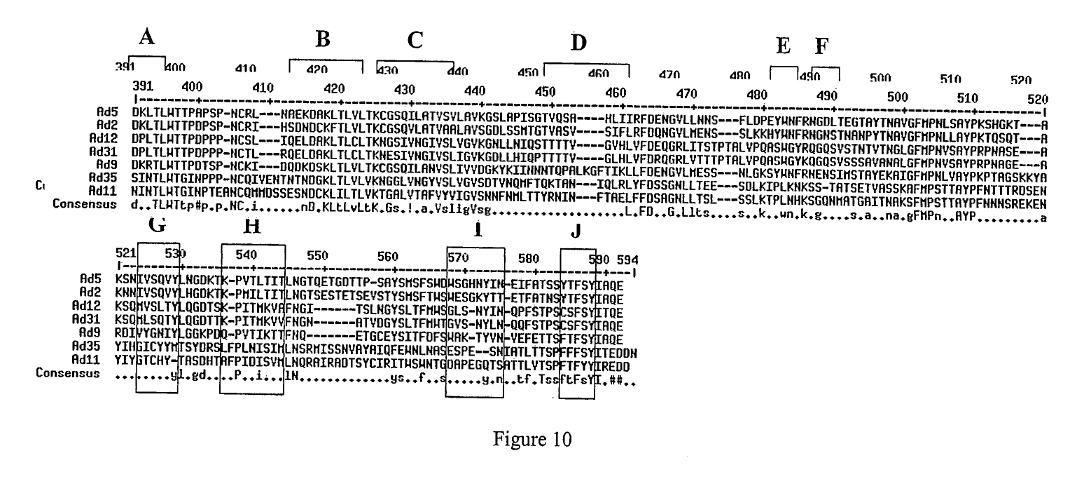

The corresponding β sheets, loops and residues of the fiber knob of a human or non-human animal adenovirus. For example, the adenoviruses can be Ad2, Ad3, Ad5, Ad7, Ad40 or Ad41 or a non-human animal virus (e.g., a canine adenovirus CAV) forming these structures can be determined by the skilled artisan. For example, referring to FIG. 10, an alignment identifying the sequences and locations of β sheets and exposed loops for exemplary adenovirus fiber knobs are shown. Additional sequence alignments can be generated, for example, using computer modeling based on the Ad5 structure deduced by Xia et al. (supra). In addition, the motifs of adenovirus fiber knobs can be identified by primary sequence alignment. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981)), by the homology alignment algorithm of Needleman and Wunsch (J. Mol. Biol. 48:443 (1970)), by the search for identity method of Pearson and Lipman (Proc. Natl. Acad. Sci. USA 85:2444 (1988)), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (J. Mol. Evol. 35:351-60 (1987)). The method used is similar to the CLUSTAL method described by Higgins and Sharp (Gene 73:237-44 (1988); CABIOS 5:151-53 (1989)). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described in Altschul et al. (J. Mol. Biol. 215:403-10 (1990)). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) can use as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin and Altschul, Proc. Natl. Acad. Sci. USA 90:5873-87 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is typically less than about 0.1, more typically less than about 0.01, and most typically less than about 0.001. Another indication that two nucleic acids are substantially identical is that the two molecules hybridize specifically to each other under stringent conditions.

Secondary structural analysis (e.g., Chou and Fasman, Biochemistry 13:222-45 (1974)) can also be conducted to identify regions of the adenovirus fiber that assume specific secondary structures. Manipulation, translation, and secondary structure prediction, open reading frame prediction and plotting, as well as determination of sequence identity and similarities, can also be accomplished using computer software programs available in the art, such as those described above. Other methods of structural analysis can also be employed. These include but are not limited to X-ray crystallography (Chou and Fasman, Biochemistry 13:222-45 (1974)) and computer modeling (Fletterick and Zoller, (eds.), “Computer Graphics and Molecular Modeling”, In Current Communications in Molecular Biology, Cold Spring Harbor Laboratory, Cold Spring Harbor, N.Y. (1986); Bordo, Comput. Appl. Biosci. 9:639-45 (1993); Bruccoleri and Karpus, Biopolymers 26:137-68 (1987); Hansen et al. Pac. Symp. Biocomput. 106-17 (1998)); Li et al., Protein Sci. 6:956-70 (1997); Sternberg and Zvelebil, Eur. J. Cancer 26:1163-66 (1990); Ring and Cohen, FASEB J. 7:783-90 (1993); and Sutcliffe et al., Protein Eng. 1:377-84 (1987)).

The mutations in the adenovirus fiber can be one or more amino acid substitutions, insertions, deletions, or combinations thereof In some embodiments, the mutations are typically induced, non-natural mutations. In exemplary embodiments, the mutations in the fiber knob can be in the exposed loop regions between the A and B, B and C, C and D, D and E, E and F, F and G, G and H, H and I, and/or I and J β sheet(s). As a result of their spatial location in the native fiber, the exposed loop residues are capable of recognizing and/or interacting directly or indirectly with a blood factor protein. In specific embodiments, mutations can be in the AB, EF and/or HI exposed loop(s), and/or in the exposed above-mentioned β sheets to reduce or ablate the affinity or avidity of the fiber for the blood factor protein. In an exemplary embodiment, an Ad5 fiber knob comprises a Y477A substitution, a deletion of amino acids 489-492 in the FG loop, and an insertion of the peptide SKCDCRGECFCD (SEQ ID NO:1) at position 547 of the HI loop. In additional embodiments, the mutations can be in the AB and EF, EF and HI, or AB and HI exposed loops. In yet other embodiments, the mutation can be in an exposed loop and in a β sheet. For example, a mutant fiber knob comprise a mutation in the AB, EF and/or HI exposed loop(s) and in the A, B, C, D, E, F, G, H, and/or J β sheet(s).

Typically, the adenovirus fiber mutant has a substantially reduced affinity or avidity for binding to the blood factor protein, but is capable of trimerizing and of binding to the penton base. Alternatively, a fiber can be modified by incorporation of a ligand at the C-terminal end which conserves its trimerization ability (see, e.g., PCT publication WO95/26412). The binding affinity of a mutant fiber for a blood factor protein can be determined, for example, by studying the infectivity or the cellular binding of the corresponding viruses, by surface plasmon resonance, or by applying the techniques of the art, such as those detailed below.

In other embodiments, the fiber shaft also can be mutated by substitution, insertion and/or deletion of amino acids. For example, a mutation in the fiber shaft can comprise deletion of all or part of a β sheet. In exemplary embodiments, the mutated fiber can comprise a short shaft of about 5, about 6 or about 7 β sheets. For example, an Ad5 fiber shaft can be mutated by deleting β sheets, or by recombining an Ad5 fiber with an Ad35 or Ad9 fiber to make a short shaft. As used herein, the term “short shaft” refers to an adenovirus shaft having a reduced length, such that clearance by the liver of an adenovirus comprising the fiber is reduced. Such clearance can be reduced, for example, as by a factor of about 10, of about 100, of about 1000, of about 10,000, or more.

In addition, in certain embodiments, a portion of a β sheet or loop (e.g., at least three amino acids) in a fiber knob and/or shaft can be deleted and replaced by residues of an equivalent loop and/or sheet derived from a fiber of a second adenovirus capable of interacting with a cellular receptor different from that recognized by the first adenovirus. The second adenovirus can be of any suitable origin, for example human or non-human animal. This makes it possible, for example, to maintain the structure of the fiber while conferring on it a host specificity corresponding to that of the second adenovirus, or of a desired target cellular receptor. As indicated in Xia et al. (supra), the cellular receptor mediating the infection by types 2 and 5 adenoviruses is different from that interacting with the types 3 and 7 adenoviruses. Thus, an Ad5 or Ad2 fiber deleted for at least 3 consecutive residues of a blood factor protein binding site can be substituted by the residues derived from an equivalent region of the Ad3 or Ad7 fiber, provided however that the inserted amino acids reduce the binding affinity for the blood factor protein.

The adenovirus fiber can be derived from an adenovirus of human or non-human origin. For example, non-human adenoviruses can include, for example, canine, avian, bovine, murine, ovine, porcine or simian origin. The adenovirus fiber also can be a hybrid and can comprise fragments of diverse origins. In certain embodiments, the fiber is derived from a human adenovirus, such as those of serotype C and, in particular, the type 2 or 5 adenoviruses (Ad2 or Ad5). The Ad2 fiber contains about 580 amino acids (aa), which sequence is disclosed, for example, by Heriss et al. (Nucleic Acid Res. 9:4023-42 (1981), the disclosure of which is incorporated by reference herein). The Ad5 fiber contains about 582 amino acids. Its sequence is reported by Chroboczek et al. (Virology 161:549-54 (1987), the disclosure of which is incorporated by reference herein). In certain other embodiments, the adenovirus fiber can originate from an animal adenovirus, such as a bovine adenovirus (e.g., the BAV-3 strain) (see, e.g., PCT publication WO 95/16048). The fiber can optionally include other modifications as compared to the native sequence, in addition to modifications of the blood factor protein binding site (see, e.g., infra).

In certain embodiments, the mutant fiber can retain substantially the same affinity for its native cellular receptor. As used herein, the term “cellular receptor” for adenoviruses refers to a cellular polypeptide(s) involved directly or indirectly in the binding of an adenovirus to its natural target cells, or in the penetration into the latter. A “native cellular receptor” refers to cellular receptor normally bound by the unmutated adenovirus fiber. A mutant fiber can have an affinity or avidity of the mutant fiber of within about 100-fold, about 50 fold, about 10 fold, about 5 fold of, or about the same as, the affinity or avidity of the wild-type fiber for the native cellular receptor.

In certain embodiments, the mutant fiber, and adenovirus comprising the fiber, is isolated. The term “isolated” refers to a nucleic acid, polypeptide or antibody that has been removed from its natural cellular environment.

In some embodiments, an adenovirus fiber according to the present invention optionally can include a ligand for a different cellular receptor, other than the native cellular receptor. The term “ligand” refers to an entity capable of recognizing and binding, typically with a high affinity, a cell surface molecule different from the native cellular receptor, provided however the ligand does not bind to a blood factor protein. Alternatively, the adenovirus fiber can have an altered tropism, in that the adenovirus fiber has a specificity for a different blood factor protein.

The ligand can be, for example, an antibody, a peptide, a hormone, a polypeptide, a sugar, or the like. The term “antibody” comprises monoclonal antibodies, antibody fragments (Fab, F(ab)₂, or the like) single-chain antibodies (scFv), heavy chain antibodies, and the like. (See generally, Harlow and Lane, Using Antibodies, A Laboratory Manual, Cold Spring Harbor Laboratory, New York (1999), the disclosure of which is incorporated by reference herein.) The target for the ligand cellular receptor can be expressed or exposed at the surface of the target cell (e.g., a cell surface marker, receptor, antigenic peptide presented by histocompatibility antigens, or the like). The addition of a ligand makes it possible to confer a new tropism toward one or more specific cell types carrying at their surface a target molecule recognized by the ligand. In certain embodiments, the regions for interaction with the natural cellular receptor can be deleted completely or partly and replaced with a ligand specific for a cell surface protein of the target cell type.

In exemplary embodiments, the ligand can bind to the class I major histocompatibility antigens, fibronectin, the CAR receptor, CD46, or any other cell surface determinant which is usually involved or which participates in the infectivity of adenoviruses. In another example, an antibody fragment (e.g., an scFv type fragment) can be inserted on the C-terminus of the fiber shaft (e.g., at the end of the β-repeating units) with the aim of modifying the specificity of infection towards cells having the target antigen (see, e.g., WO 94/10323). In yet another example, tumor necrosis factor (TNF) or a TNF-receptor binding fragment thereof, can be inserted in an Ad5 chimeric fiber so as to facilitate interaction of the adenovirus with the cellular receptor for TNF (see, e.g., U.S. Pat. No. 5,543,328). In yet another example, an Ad5 native fiber can be fused at its C-terminal end with an ApoE peptide, allowing binding to the LDL (for low density lipoprotein) receptor present at the surface of hepatic cells. In a further example, a chimeric fiber obtained by replacing part of the native fiber and with an equivalent part of an adenoviral fiber of another serotype can be modified by inserting at the C-terminal end a peptide RGD which is specific for vitronectin (see, e.g., PCT publication WO96/26281).

A ligand also can be used to target, for example, a tumor cell, an infected cell, a particular cell type or a category of cells carrying a specific surface marker. For example, if the host cell to be targeted is a cell infected with the HIV virus (Human Immunodeficiency Virus), the ligand can be a fragment of antibody against fusin, the CD4 receptor, against an exposed viral protein (e.g., envelope glycoprotein) or a part of the TAT protein of the HIV virus (e.g., extending from residues 37 to 72) (see, e.g., Fawell et al., Proc. Natl. Acad. Sci. USA 91:664-68 (1994)). In the example of a tumor cell, the ligand can recognize an antigen specific for tumors (e.g., a tumor specific antigen, such as for example the MUC-1 protein in the case of breast cancer, some epitopes of the E6 or E7 proteins of the papillomavirus HPV, or the like) or an antigen that is overexpressed on tumor cells (e.g., the receptor for IL-2 overexpressed in some lymphoid tumors; Gastrin Releasing Peptide (GRP) which is overexpressed in lung carcinoma cells (Michael et al., Gene Therapy 2:660-68 (1995) and in pancreas, prostate and stomach tumors; or the like). T lymphocytes can be targeted, for example, using a ligand for the T cell receptor. In general, the ligands that can be used are widely described in the literature and can be cloned by standard techniques. It is also possible to synthesize ligands by the chemical route and to couple them to an adenovirus fiber (see, e.g., Hunkapiller et al., Nature 310:105-11 (1984); Stewart and Young, Solid Phase Peptide Synthesis, 2nd Ed., Pierce Chemical Co., Rockford, Ill., (1984)). Further, the coupling of galactosyl residues can confer a hepatic or other specificity because of the interaction with the asialoglycoprotein or other receptors.

The ligand also can be chemically coupled to the adenovirus fiber, or the adenovirus. The sequences encoding the ligand also can be inserted into the adenoviral genome, in particular, into sequences encoding the modified fiber. The insertion can take place at any suitable site in the adenoviral genome. In certain embodiments, the site of insertion is upstream of the stop codon at the C-terminal end or in place of the deleted residues with the coding region of the fiber or the fiber knob. Ligand sequences also can be inserted into other adenoviral sequences, such as, for example, those encoding another capsid protein, such as the hexon or the penton.

In another aspect, a DNA fragment encoding a mutant adenovirus fiber, and a vector for expressing such a DNA fragment, are provided. The necessary transcriptional and translational signals can also be supplied by the native adenoviral nucleic acids and/or its flanking regions, or can be heterologous. The DNA fragment can be, for example, an expression cassette. Such an expression cassette optionally can include a heterologous promoter operatively linked to a DNA fragment encoding a mutant adenovirus fiber. The vector can be, for example, a plasmid or virus, integrative or otherwise.

The DNA fragment, expression cassette and/or vector also can be combined with one or more substances capable of improving the transfection efficiency and/or the stability of the fragment, cassette or vector. Such substances include, for example, polymers, lipids (e.g., cationic lipids), liposomes, nuclear proteins and neutral lipids.

In a related aspect, a human or non-human, animal adenovirus is provided that comprises a human or non-human, animal mutated fiber having a reduced or ablated blood factor protein binding site. The mutated fiber is typically present at the surface of the virus. The mutant fiber can be encoded by the adenovirus or provided in trans by a host cell line (e.g., a cell line expressing the mutant fiber). The adenovirus optionally can comprise a ligand displayed on the surface of the virus. In certain embodiments, the specificity of binding of the adenovirus to its natural cellular receptor is not significantly reduced. In other embodiments, the specificity of binding of the adenovirus to its natural cellular receptor is significantly reduced or abolished. The specificity of the binding can be evaluated by studies of cellular attachment carried out, for example, in the presence of labeled viruses (for example labeled with ³H-thymidine according to the technique of Roelvink et al. (J. Virol. 70:7614-21 (1996))), by studies of infectivity of cells which are permissive or which express the surface molecule targeted by the ligand, or the like.

In certain embodiments, the adenovirus can be a recombinant and replication-defective adenovirus (i.e., incapable of autonomously replicating in a host cell). Such a replication-deficient host cell can include, for example, a mutation or deletion of one or more essential viral regions, such as, for example, all or part of the E1 region and/or E3 region. The genome of an adenovirus optionally can include additional deletions or mutations affecting other regions, such as, for example, the E2, E4 and/or L1-L5 regions, including complete deletion of the virus coding sequences and replacement with non-adenovirus DNA (so called “helper-dependent” vectors).

The adenovirus optionally can be a recombinant adenovirus and comprise one or more genes of interest placed under the control of the elements necessary for their expression in a host cell. The gene of interest is typically a human or non-human heterologous gene (i.e., a non-adenoviral gene). The gene of interest can be, for example, genomic, cDNA (complementary DNA), a hybrid or chimeric gene (e.g., a minigene lacking one or more introns), or the like. It can be obtained, for example, by conventional molecular biology techniques and/or by chemical synthesis. A gene of interest can encode, for example, an antisense RNA, a ribozyme or an mRNA that can be translated into a polypeptide of interest. A polypeptide of interest can be, for example, a cytoplasmic, membrane, secreted or other type of protein. Further, the polypeptide of interest can be, for example, a polypeptide as found in nature, a chimeric polypeptide obtained from the fusion of sequences of diverse origins, or of a polypeptide mutated relative to the native sequence having improved and/or modified biological properties.

In certain embodiments, a gene of interest can encode, for example, one of the following polypeptides: cytokines or lymphokines (α-, β- or -γ-interferons, interleukins (e.g., IL-2, IL-6, IL-10 or IL-12), tumor necrosis factors (TNF), colony stimulating factors (e.g., GM-CSF, C-CSF, M-CSF, or the like)); cellular or nuclear receptors (e.g., those recognized by pathogenic organisms (e.g., viruses, bacteria or parasites)); proteins involved in a genetic diseases (e.g., factor VII, factor VIII, factor IX, dystrophin or minidystrophin, insulin, CFTR protein (Cystic Fibrosis Transmembrane Conductance Regulator)); growth hormones (e.g., insulin, hGH or the like); enzymes (e.g., urease, renin, thrombin, or the like); enzyme inhibitors (e.g., α1-antitrypsin, antithrombin III, viral protease inhibitors, or the like); polypeptides with antitumor effect (e.g., which are capable of at least partially inhibiting the initiation or the progression of tumors or cancers), such as antibodies, inhibitors acting on cell division or transduction signals, products of expression of tumor suppressor genes (e.g., p53 or Rb), proteins stimulating the immune system, or the like); proteins of the class I or II major histocompatibility complex or regulatory proteins acting on the expression of the corresponding genes; polypeptides capable of inhibiting a viral, bacterial or parasitic infection or its development (e.g., antigenic polypeptides having immunogenic properties, antigenic epitopes, antibodies, transdominant variants capable of inhibiting the action of a native protein by competition, or the like); toxins (e.g., herpes simplex virus 1 thymidine kinase (HSV-1-TK), ricin, cholera toxin, diphtheria toxin, or the like) or immunotoxins; markers (β-galactosidase, luciferase, Green Fluorescent Protein, or the like); polypeptides having an effect on apoptosis (e.g., inducer of apoptosis: Bax, or the like, inducer of apoptosis Bc12, Bclx, or the like); cytostatic agents (e.g., p21, p16, Rb, or the like); apolipoproteins (e.g., apoE or the like); superoxide dismutase, catalase, nitric oxide synthase (NOS); growth factors (e.g., Fibroblast Growth Factor (FGF), Vascular Endothelial Cell Growth Factor (VEGF), insulin, or the like), or others genes having therapeutic or research interest. It should be noted that this list is not limiting and that other genes can also be used. In certain embodiments, the polypeptide of interest is not a marker (e.g., β-galactosidase, luciferase, Green Fluorescent Protein, or the like).

The adenovirus optionally can include a selectable gene which makes it possible to select or identify the infected cells. Suitable selectable genes include, for example, neo (encoding neomycin phosphotransferase), dhfr (Dihydrofolate Reductase), CAT (Chloramphenicol Acetyl transferase), pac (Puromycin Acetyl-Transferase), gpt (Xanthine Guanine Phosphoriboxyl Transferase), or the like. In other embodiments, the adenovirus is free of selectable genes.

The adenovirus optionally can include elements necessary for the expression of a gene of interest in a host cell. Such elements include, for example, elements facilitating transcription of the gene into RNA and/or the translation of an mRNA into a protein. Suitable promoters include, for example, those of eukaryotic or viral origin. Suitable promoters can be constitutive or regulatable (e.g., inducible). A promoter can be modified to increase promoter activity, suppress a transcription-inhibiting region, make a constitutive promoter regulatable, or the like, introduce a restriction site, or the like. Examples of suitable promoters include, for example, the CMV (Cytomegalovirus) viral promoter, the RSV (Rous Sarcoma Virus) viral promoter, the promoter of the HSV-1 virus TK gene, the early promoter of the SV40 virus (Simian Virus 40), the adenoviral MLP promoter, the eukaryotic promoters of the murine or human genes for PGK (Phospho Glycerate kinase), MT (metallothionein), al-antitrypsin and albumin (liver-specific), immunoglobulins (lymphocyte-specific), a tumor-specific promoter (e.g., α-fetoprotein AFP (see, e.g., Ido et al., Cancer Res. 55:3105-09 (1995)); MUC-1; prostate specific antigen (PSA) (see, e.g., Lee et al., J. Biol. Chem. 271:4561-68 (1996)); and flt1 specific for endothelial cells (e.g., Morishita et al., J. Biol. Chem. 270:27948-53 (1995)).

A gene of interest can also include additional elements for the expression (e.g., an intron sequence, a signal sequence, a nuclear localization sequence, a transcription termination sequence, a site for initiation of translation of the IRES type, or the like), for its maintenance in the host cell, or the like.

Also provided are methods of preparing a human or non-human adenovirus according to the present invention. Such methods can include, for example, transfecting the genome of the adenovirus (encoding a mutant adenovirus fiber) into an appropriate cell line and culturing the transfected cell line under appropriate conditions in order to allow the production of the adenovirus. The adenovirus optionally can be recovered from the culture. In certain embodiments, the adenovirus is substantially purified.

The cell line can be selected according to the deficient functions in the adenovirus, as applicable. A complementation host cell line capable of providing in trails the deficient function(s) can be used. In certain embodiments, the 293 line is used for complementing the E1 function (see, e.g., Graham et al., J. Gen. Virol. 36:59-72 (1977)). A complementation host cell line also can complement multiple adenoviral gene deficiencies, such as, for example, a deficiency of the E1 and E2 or E4. In certain embodiments, a helper virus can be used to complement the defective adenovirus in a host cell. Methods of propagating defective adenoviruses are known in the art (see, e.g., Graham and Prevec, Methods in Molecular Biology (ed. E. J. Murey, The Human Press Inc.), vol. 7, p. 190-128 (1997)). The adenoviral genome also can be reconstituted in vitro in, for example, Escherichia coli (E. coli) by ligation and/or by homologous recombination.

In another aspect, a cell line is provided comprising, either in a form integrated into the genome or in the form of an episome, a DNA fragment encoding a mutant adenovirus fiber. The cell line is optionally placed under the control of the elements allowing its expression. The cell line optionally can be capable of complementing an adenovirus deficient for one or more functions, such as, for example, those encoded by the E1, E2, E4 and/or L1-L5 regions.

In certain embodiments, such a cell line can be used to prepare an adenovirus whose genome lacks all or part of the sequences encoding the fiber (so as to produce a nonfunctional fiber). For example, the genome of an adenovirus can be transfected into a cell line and the transfected cell line cultured under appropriate conditions in order to allow the production of the adenovirus comprising the mutant adenovirus fiber. The mutated adenovirus fiber can be provided in trans. The adenovirus optionally can be recovered from the culture of the transfected cell line and/or substantially purified.

Further provided are a host cell infected with an adenovirus according to the present invention or capable of being obtained by a method according to the present invention. The infected host cell, can be, for example, a mammalian cell, such as a human cell, or a non-human, animal cell. An infected host cell also can be, for example, a primary or tumor cell and of any suitable origin, for example, of hematopoietic (e.g., a totipotent stem cell, leukocyte, lymphocyte, monocyte or macrophage, or the like), muscle (e.g. a satellite cell, myocyte, myoblast, smooth muscle cell), cardiac, nasal, pulmonary, tracheal, hepatic, epithelial or fibroblast origin.

Further provided are pharmaceutical compositions comprising, as therapeutic or prophylactic agent, a host cell or an adenovirus, in combination with a pharmaceutically acceptable carrier. In certain embodiments, the composition can be used for preventive and/or treatment of diseases, such as genetic diseases (e.g., hemophilia, cystic fibrosis, diabetes, Duchenne's myopathy or Becker's myopathy, or the like), cancers, such as those induced by oncogenes or viruses, viral diseases, such as hepatitis B or C and AIDS (acquired immunodeficiency syndrome resulting from HIV infection), recurring viral diseases, such as viral infections caused by the herpesvirus and cardiovascular diseases including restenoses.

A pharmaceutical composition can be manufactured by any suitable means. For example, a therapeutically effective quantity of the therapeutic or prophylactic agent can be combined with a carrier such as a diluent. The composition also can include an adjuvant, a pharmaceutically acceptable excipient(s), a stabilizer, a preservative, a solubilizer, or the like. In certain embodiments, the pharmaceutical composition can be disposed in a saline, nonaqueous or isotonic solution for an injectable administration. The pharmaceutical composition can be provided in liquid or dry form (e.g., a lyophilisate, or the like) or any other suitable galenic form.

Further provided is a kit comprising a pharmaceutical composition and another component, such as, for example, sterile water of injection, a saline, nonaqueous or isotonic solution, a syringe, instructions, or the like. The pharmaceutical composition in the kit optionally can be lyophilized. The pharmaceutical composition can include, for examples a recombinant adenoviral vector comprising a mutant adenovirus fibers (having a reduce or ablated blood factor binding site) and a heterologous gene.

The pharmaceutical composition can be administered by, for example, local, systemic or aerosol route, by intragastric, subcutaneous, intracardiac, intra-muscular, intravenous, intraperitoneal, intratumor, intrapulmonary, intranasal or intracheal route. The administration can take place in a single dose or repeated once or several times after a certain time interval. The appropriate route of administration and the appropriate dosage vary according to various parameters, for example, the individual or patient to be treated or the gene(s) of interest to be transferred. In exemplary embodiments, the viral particles can be formulated in the form of doses of between about 10⁴ and about 10¹⁴ pfu (plaque-forming units), between about 10⁵ and about 10¹³ pfu, and between about 10⁶ and 10¹² pfu.

In additional embodiments, the therapeutic or prophylactic use of an adenovirus or of a host cell according to the present invention is provided for the preparation of a medicament intended for the treatment of the human or animal body by gene therapy. The medicament can be administered directly in vivo (e.g., by intravenous injection, into an accessible tumor, into the lungs by aerosol, or the like). The medicament also can be administered using an ex vivo strategy, such as, for example, by collecting cells from the patient (e.g., bone marrow stem cells, peripheral blood lymphocytes, muscle cells, or the like), transfecting or infecting the cells in vitro according to prior art techniques and re-administering them to the patient.

Further provided are methods of treatment according to which a therapeutically effective quantity of an adenovirus or of a host cell is administered to a patient requiring such a treatment.

In yet another aspect, a method is provided of identifying a blood factor protein binding an adenoviral fiber. The method generally includes providing a nucleic acid (e.g., a DNA or RNA sequence) encoding an adenovirus fiber. The nucleic acid can encode the entire fiber or a fragment thereof, such as for example, a fiber shaft or knob. The fiber is expressed and contacted with a suitable source of blood factor protein. Blood factor protein can be obtained, for example, for plasma or from recombinant sources. The blood factor protein is contacted with the fiber for sufficient time to allow the formation of blood factor protein/fiber complex. The complex can be isolated and the resulting analyzed for the presence of a blood factor protein. To facilitate isolation of the fiber, it can optionally be affinity labeled. Alternatively, the fiber can be purified using, for example, an antibody against the fiber. The blood factor protein can be identified, for example, by gel electrophoresis, mass spec, immunological methods, or the like.

In a further aspect, a method of identifying blood factor binding sites are provided. For example, the nucleic acid encoding the adenovirus fiber, or a fragment thereof, can be mutated, the mutation resulting in a mutation in the encoded adenovirus fiber. Suitable mutation methods include random and site-directed methodologies. The mutated nucleic acids can be expressed and the fibers contacted with a suitable source of blood factor protein. Blood factor protein can be obtained, for example, for plasma or from recombinant sources. The blood factor protein is contacted with the fiber for sufficient time to allow the formation of blood factor protein/fiber complex. The complex can then be isolated and the resulting analyzed for the presence of a blood factor protein. To facilitate isolation of the fiber, it can optionally be affinity labeled (e.g., a polyhistidine label). Alternatively, the fiber can be purified using, for example, an antibody against the fiber. The binding of the blood factor protein to the fiber can be identified, for example, by gel electrophoresis, mass spec, immunological methods, or the like. (See generally Ausubel et al. (1999) supra; Sambrook et al., Molecular Cloning: A Laboratory Manual, 3d Ed., Cold Spring Harbor Laboratory Press, New York (2001), which are incorporated by reference herein.)

The following examples are offered to illustrate, but not to limit the claimed invention.

### EXAMPLES

### Example 1

In this study, a new pathway is described that is utilized by Ad for infection of liver. The results demonstrate that Ads, which were unable to infect hepatocytes in vitro due to an inability to interact with CAR, efficiently infected hepatocytes in vivo through binding of viral particles to blood factors, in particular to coagulation FIX.

Methods

Cells and Viruses. 293 cells were from Microbix (Toronto, Canada). CHO-K1 (CCL-61) and CHO-pgsA745 (CRL-2242) cells were from the ATCC. Plated primary human hepatocytes were from BioWhittaker (Walkersville, Md.). MEF Lrp^{+/+} and MEF Lrp^{-/-} cells were kindly provided by Dr. Michael Gotthardt (MDC, Berlin, Germany). All cell lines were grown on Dulbecco's Modified Eagles Medium, supplemented with 10% fetal bovine serum. 293-DH26 cells were obtained by stable transfection of 293 cells with plasmid pDH.2 expressing the membrane anchored scFv, recognizing a 6-His tag (Douglas et al., Nat. Biotechnol. 17:470-75 (1999)). Primary mouse hepatocytes were isolated by collagenase perfusion (Lieber et al., Hum. Gene Ther. 6:5-11 (1995)) and cultured in PRIMARIA™ dishes (Corning Corp., Acton, Mass.) in William's E Medium, supplemented with 10% fetal bovine serum. Ad5 is described as Ad5Luc1 by Krasnykh et al. (J. Virol. 75:4176-83 (2001)). The detailed structure of Ad5FF/6His is described by Krasnykh et al. (J. Virol. 75:4176-83 (2001)), where this vector was designated as Ad5LucFF/6H. Ad5 and Ad5FF/6His express luciferase; Ad5L1 and Ad5F* express luciferase and GFP; and Ad5L2 and Ad5/35L express β-galactosidase from identical expression cassettes. Ad5L1 and Ad5F* are described elsewhere as Ad5GFPLuc and AdGFPLucY477Ax6H, respectively (Alemany et al., Gene Ther. 8:1347-53 (2001)). Ad5F* contains a mutation that compromises CAR binding. The Ad5mut virus contains the following mutations: the Y477A mutation (Alemany et al., Gene Ther. 8:1347-53 (2001)), a deletion of amino acids 489-492 (TAYT) in the FG loop, a peptide insertion (SKCDCRGECFCD; SEQ ID NO:8) into position 547 of the HI loop, and a C-terminal 6-histidine tag as described in Ad5F* (Alemany et al., Gene Ther. 8:1347-53 (2001)). The detailed structures of Ad5L and S, Ad5/35L and S, and Ad5/9L and S are described in detail by Shayakhmetov et al. (J. Virol. 74:10274-86 (2000)). All viruses were propagated on 293 or 293-DH26 cells, purified and titered for genomes and plaque forming units as described by Shayakhmetov et al. (J. Virol. 74:10274-86 (2000)).

Ad infection in vitro. 293 and 293-DH26 cells (2.5×10⁵) were infected with Ad5 and Ad5FF/6His at MOIs of 0, 40, or 400 virus particles per cell for 2 hours. Twenty four hours post-infection, luciferase activity was measured in cell lysates. 2.5×10⁵ CHO-Kl, CHO-pgsA745, MEF Lrp^{+/+}, MEF Lrp^{-/-} cells and primary human hepatocytes were infected at an MOI of 1000 viral particles/cell with or without FIX (3 U/ml) in 300 μl saline. Two hours later, the virus-containing saline was replaced by growth media. Reporter gene activity was analyzed 48 hours later. In competition studies human lactoferrin (0.5 mg/ml) or heparin (10 U/ml) was used.

Ad infection in vivo. All study procedures were conducted in accordance with the institutional guidelines set forth by the University of Washington. Mice were housed in specific pathogen-free facilities. Unless indicated otherwise, Ads were injected into the portal vein through a permanently placed catheter at a dose of 1×10¹¹ viral particles/mouse in 200 μl saline.

To analyze the role of blood factors in liver transduction by Ads, in the setting “with blood,” fifteen minutes after Ad infusion, liver was flushed with HBSS followed by collagenase perfusion to isolate and culture hepatocytes for analysis of reporter gene expression. Cell preparations had less than 5% contamination with other liver cell types (Seglen, J. Toxicol. Environ. Health 5:551-60 (1979)). In settings “without blood,” the v. porta and v. cava inferior were canulated and blood was flushed from the liver through the portal vein. Then, virus (2.5×10¹⁰ viral particles/mil) in 8 ml of saline was infused through the portal vein and the circulation between v. porta and v. cava was closed allowing asanguinous isolated liver perfusion (at 37° C.). Thirty minutes after virus application, hepatocytes were isolated by collagenase perfusion. In both settings, reporter gene expression in plated hepatocytes was analyzed 48 hours post infection.

In vivo competition studies were done by pre-injecting polymerized BSA (pBSA) at 1 mg/mouse, asialofetuin (ASF) at 0.5 mg/mouse, human LDL (hLDL) at 0.5 mg of protein/mouse, lactoferrin at 1 mg/mouse, or saline (as a control) 5 minutes before virus administration into the portal vein. Reporter gene expression was analyzed in hepatocytes as described above. Heparinase I (30 U per mouse) was administrated into the tail vein 30 minutes before virus injection.

Southern blot analysis. Seventy two hours post injection, liver DNA (1 μg) was analyzed for viral DNA by Southern blotting as described earlier (Shayakhmetov et al., Cancer Res. 62:1063-68 (2002)). Equivalent loading was assessed by hybridization of the membranes with a mouse β-glucuronidase specific probe.

Surface Plasmon Resonance and protein cross-linking assays. A PCR fragment containing the Ad35 knob domain was cloned into pQE30 plasmid (Qiagen, Calif.); recombinant Ad35 knob protein was expressed in E. coli and purified as described elsewhere for Ad5 (Krasnykh et al., J. Virol. 75:4176-83 (2001)). Ten μg of human FIX were immobilized on a Surface Plasmon Resonance (SPR) sensor and a solution containing purified recombinant Ad5, Ad35 or Ad5mut fiber knob domains (1 μg/ml) was perfused through the sensor chamber. Data were collected and analyzed as described elsewhere (Naimushin et al., Biosens. Bioelectron. 17:573-84 (2002)). For in vitro cross-linking studies, 10 μg of recombinant FIX was mixed with 5, 10, or 20 μg of purified Ad5 knob, Ad35 knob, or BSA. Thirty minutes post incubation at room temperature, 2 μl of the BS3 cross-linking reagent (Pierce) was added to the protein mixtures, followed by another incubation for 30 minutes. Samples were run on a 10% SDS-PAGE gel, transferred onto nitrocellulose membrane, and developed with anti-FIX rabbit polyclonal antibodies.

Results

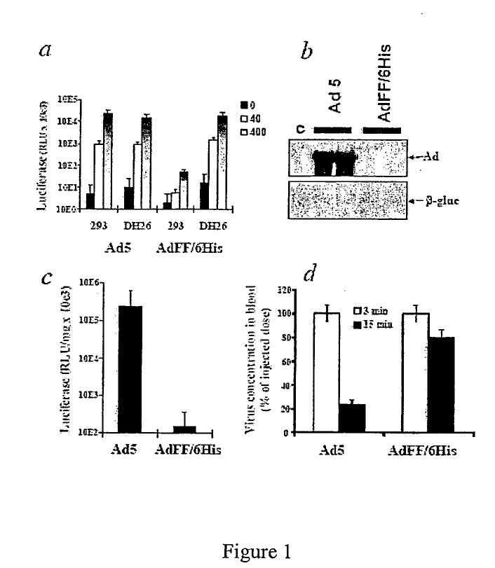

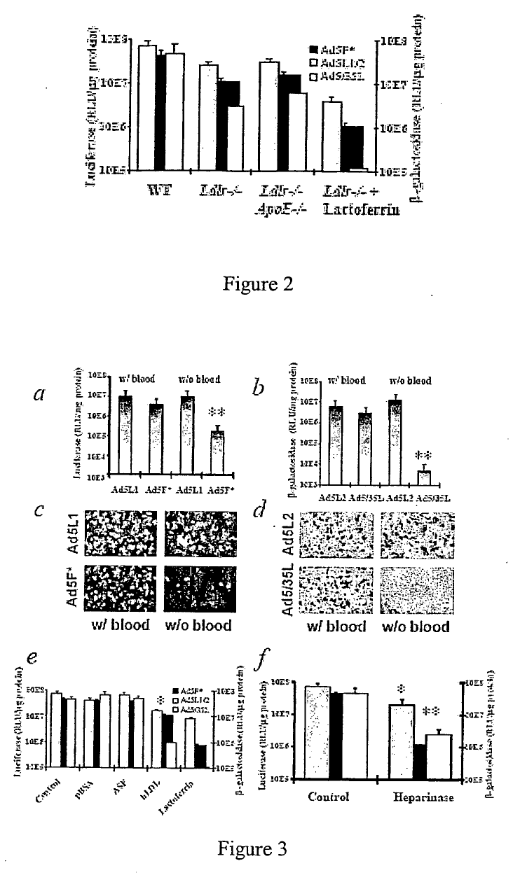

Ad infection of liver is mediated by the fiber knob domain. To delineate which capsid protein is responsible for Ad tropism in vivo, liver transduction by Ad vectors containing fibers (Ad5) was compared to transduction by Ad vectors lacking native fibers (AdFF/6His) (FIG. 1). AdFF/6His has the majority of the Ad5 fiber deleted and replaced with a T4 phage-derived fibritin molecule containing a 6-His tag as a novel receptor recognition moiety. While AdFF/6His infected 293 cells poorly, likely due to the lack of a CAR-interacting fiber, this vector infected 293-DH26 cells, which express both the artificial receptor for the histidine tag and CAR, with comparable efficiency to Ads (FIG. 1a). After intravenous injection of AdFF/6His into mice, the levels of hepatic vector DNA (determined by quantitative Southern blot) and transgene expression were more than three orders of magnitude lower than for Ad5 (FIGS. 1b and c). Preferential sequestration of AdFF/6His in other organs appeared not to account for the inefficient liver transduction (FIG. 2) because 80% of injected AdFF/6His was detected in the blood 15 minutes after injection compared to 20% for Ad5 (FIG. 1d). These data suggest that the fiber is the main viral protein that directs Ad5 to the liver.

The Ad fiber can be divided into three domains, the N-terminal tail, which provides the contact with penton base, the rod-like shaft, and the C-terminal globular fiber knob. The Ad5 fiber knob contains the CAR-binding amino acid residues (Roelvink et al., Science 286:1568-71 (1999)). Furthermore, the third repeat of the Ad5 fiber shaft was recently suggested as a receptor binding motif for heparin sulfate glycosaminoglycans (Dechecchi et al., Virology 268:382-90 (2000); Smith et al., Human Gene Therapy 14:777-87 (2003)). To analyze in more detail the structural domains within the fiber that allow for liver transduction, a series of Ad5-capsid based vectors containing long- or short-shafted fibers with knobs derived from Ad5, Ad9 (subgroup C and D, CAR-interacting) (Roelvink et al., J Virol. 72:7909-15 (1998)) and Ad35 (subgroup B, non-CAR-interacting) (Shayakhmetov et al., J. Virol. 74:2567-83 (2000)) were used. Notably, the putative receptor-binding motif described by Dechecchi et al. and Smith et al. is not contained within short fiber vectors (Dechecchi et al., Virology 268:382-90 (2000); Smith et al., Human Gene Therapy b 14:777-87 (2003)). For all three vectors with long-shafted fibers, the amounts of vector DNA present in the liver after intravenous injection were more than 100 fold greater than in other organs. For the short-shafted vectors, the amount of hepatic vector DNA varied over a 20-fold range. The variability between the short-fiber vectors, which differed only in their knob domains suggests that the knob and not the shaft domain mediates liver transduction in vivo. The higher transduction rates with long-shafted vectors indicate that interaction with liver cells in vivo could be sensitive to steric hindrance and/or electrostatic repulsion between the viral capsid and the cell surface (Shayakhmetov et al. J. Virol. 74:10274-86 (2000); Chiu et al., J. Virol. 75:5375-80 (2001)).

Next, the intrahepatic distribution of long-shafted Ad5L and Ad5/35L vectors in vivo on liver sections was studied. Early after Ad infusion, the vast majority of Ad5L and Ad5/35L particles, conjugated with the fluorophore Cy-3, were found in association with Kupffer cells. Kupffer cells were identified by positive staining for CD45 (Worgall et al., Hum. Gene Ther. 8:37-44 (1997)) and for the macrophage specific marker F4/80 and based on their sensitivity to gadolinium chloride (Hardonk et al., J. Leukoc. Biol. 52:296-302 (1992)). At 1 hour p.i., only a small portion of the administrated particles were present in hepatocytes. Since the incoming Cy-3 labeled capsid proteins will be degraded over time, viral distribution at day 3 p.i. was assessed based on Ad-mediated transgene expression. At this time point, GFP or β-galactosidase expression were only detectable in hepatocytes and not in the Kupffer cells or CD31+ vascular endothelial cells.

Surprisingly, although the long shafted non-CAR interacting Ad5/35 vector efficiently transduced hepatocytes in vivo, it could neither bind to nor transduce primary mouse hepatocytes in vitro (Shayakhmetov et al., Cancer Res. 62:1063-68 (2002)). This implies the existence of a novel mechanism for Ad infection of hepatocytes i71 vivo. It was hypothesized that factors present only in vivo, specifically blood factor(s), serve as a bridge between the virus knob and the cell surface, allowing for efficient Ad infection of liver cells in vivo.

Blood factors are required for CAR-independent hepatocyte infection in vivo. To assess the contribution of blood factors to liver transduction, an in situ perfusion technique was employed that allows for vascular exclusion of the liver and analysis of gene delivery to hepatic cells in the absence of blood (see Methods (supra); Vrancken Peeters et al., Biotechniques 20:278-85 (1996); Branchereau et al., Hum. Gene Ther. 5:803-08 (1994)). In vivo transduction was analyzed for the native CAR-binding Ad5-based vectors, Ad5L1 (expressing GFP and luciferase) and Ad5L2 (expressing β-galactosidase), and compared to transduction of non-CAR binding vectors, Ad5F* (expressing GFP and luciferase) and Ad5/35L (expressing β-galactosidase). Ad5F* is a long-shafted Ad5-based vector with a single point mutation in the fiber knob domain (aa 477 Y→A) that compromises CAR binding (Alemany et al., Gene Ther. 8:1347-53 (2001)). Upon vector infusion in the presence of blood, reporter gene expression in hepatocytes was comparable for CAR interacting (Ad5L1/2) and non-CAR interacting vectors (Ad5F* and Ad5/35L) (FIG. 3a and b). However, liver perfusion in the absence of blood resulted in two to three orders of magnitude less efficient hepatocyte transduction with non-CAR interacting vectors, compared to corresponding CAR binding controls. Ad5L1/2 efficiently transduced hepatocytes in the absence of blood, most likely through interaction with CAR. In conclusion, these data demonstrate that liver uptake of Ad5 vectors occurs through two different mechanisms: via interaction with CAR, and a novel, CAR-independent mechanism mediated by blood factors. In the following studies, the non-CAR interacting vectors, Ad5/35L and Ad5F*, were employed as tools to investigate the blood-factor mediated pathway.

Heparan sulfate proteoglycans are the major hepatocellular receptors for Ad infection in vivo. To identify the cellular receptor(s) involved in binding and uptake of the complex(es) formed between Ad and putative blood factor(s), a number of ligands for known hepatocellular receptors were injected intravenously into mice to test whether they could compete with Ad infection in vivo. These ligands included polymerized BSA to saturate the scavenger receptor SR-BI (Takami et al., J. Biochem. (Tokyo) 111:714-21 (1992)), asialofetuin to saturate the asialo glycoprotein receptor (AGPR) (Windler et al., Biochem. J. 276:79-87 (1991)), human LDL to saturate the LDL receptor (LDLR) (Brown et al., Science 232:34-47 (1986)), and lactoferrin to saturate the LDL-related receptor (LRP) as well as heparan sulfate proteoglycans (HSPG) (Herz et al., Cell 71:411-21 (1992); Ji et al., Arterioscler. Thromb. 14:2025-31 (1994)) (FIG. 3e). Among the ligands analyzed, only lactoferrin strongly inhibited infection with Ad5F* (>50× reduction in reporter-gene activity) and Ad5/35L (>600× reduction), whereas the efficiency of Ad5L infection was reduced by only 8 fold (p<0.001). Although the LDL also reduced Ad infection, there was no significant difference in liver transduction of Ldlr^{-/-} knockout (Yu et al., J. Clin. Invest. 107:1387-94 (2001)) and wild-type mice (see FIG. 2), indicating that LDLR does not play a major role in Ad infection in vivo.

Lactoferrin binds to both LRP and HSPG (Ji et al., Arterioscler. Thromb. 14:2025-31 (1994)). Because the LRP knockout in mice is lethal (Herz et al., Cell 71:411-21 (1992)), the role of HSPG as a potential receptor for Ad infection in vivo was tested. It has been shown that injection of heparinase in vivo dramatically reduces clearance of proteins from blood whose catabolism depends on HSPG (Ji et al., J. Lipid Res. 36:583-92 (1995)). According to the protocols used in these studies, wild-type mice were injected with heparinase followed by virus administration (FIG. 3f). The reporter gene activities in hepatocytes harvested and cultured after Ad5F* and Ad5/35L infection were almost two orders of magnitude lower in mice pre-treated with heparinase compared to untreated controls (P<0.0001). At the same time transduction with CAR-interacting Ad5L vectors was significantly less affected by heparinase treatment (5-fold decrease in reporter gene expression, P<0.01). These data indicate that HSPGs represent a major cellular receptor, enabling the non-CAR interacting vectors, Ad5F* and Ad5/35L, to transduce hepatocytes in the presence of blood.

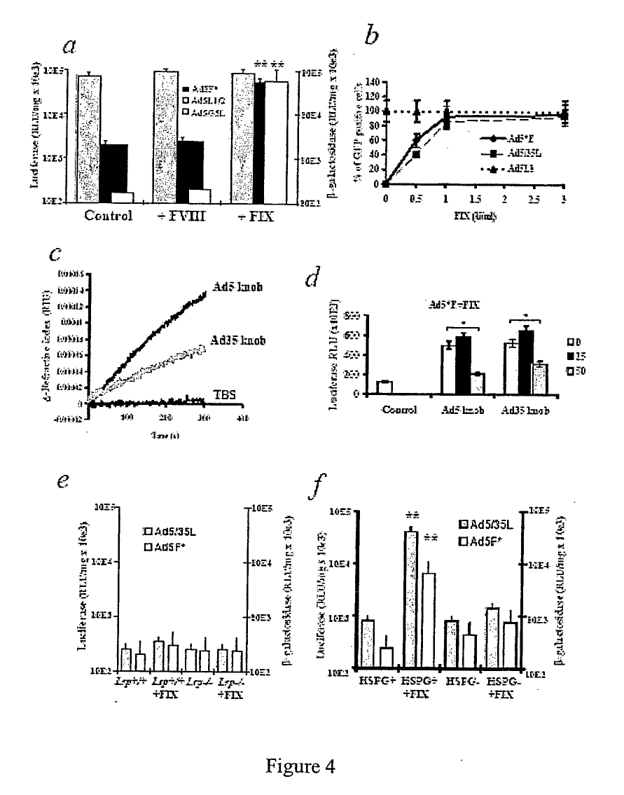

Factor IX serves as a bridge between Ad and hepatocytes in vivo. HSPG and LRP interact in vivo with a large variety of structurally unrelated ligands, including ApoE-containing lipoproteins (VLDL, HDL, IDL), chylomicrons, activated blood clotting factors (FVIIIa, FIXa, FXa, TFPI), complement C3, antithrombin III, and the majority of circulating proteinase/proteinase inhibitor complexes (Herz et al., J. Clin. Invest. 108:779-84 (2001)). The involvement of ApoE-containing lipoproteins or chylomicrons in Ad infection in vivo was excluded by administering viruses into Ldlr^{-/-} ApoE^{-/-} double-knockout mice (Ishibashi et al., Proc. Natl. Acad. Sci. USA 91:4431-35 (1994)), where reporter gene activities were not further reduced compared to control mice (FIG. 2). To screen the other HSPG/LPR ligands for their ability to confer CAR-independent Ad infection, primary mouse hepatocytes and human HepG2 hepatoma cells (known to express HSPG and LRP (Ji et al., J. Biol. Chem. 268:10160-67 (1993)) were infected with Ad5F* and Ad5/35 in the presence or absence of purified blood proteins. From all factors analyzed, only recombinant human FIX, in a calcium dependent manner, allowed for infection of hepatocytes in vitro with the non-CAR interacting vectors at a level comparable to that of Ad5L1 infection. To test whether this finding was relevant to Ad infection in vivo, mouse livers were perfused with Ad5L1/2, Ad5F*, or Ad5/35L virus containing saline with and without supplementation with FIX and human FVIII (as a negative control). Addition of FVIII (FIG. 4a) resulted only in a modest increase in infectivity of Ad5F* and Ad5/35L vectors. In contrast, supplementation of saline with FIX allowed for the transduction of hepatocytes with the non-CAR interacting Ad5F* and Ad5/35L viruses to levels comparable with Ad5L1/2 (P<0.0001). Perfusion of mice with increasing concentrations of human recombinant FIX resulted in a dose-dependent increase in hepatocyte transduction with Ad5F* and Ad5/35L viruses (FIG. 4b).

As demonstrated above, within the Ad capsid, the fiber knob is the structural moiety that mediates Ad infection in vivo. To demonstrate a direct interaction between Ad fiber knob and FIX, the following three studies were performed involving purified recombinant trimeric Ad5 and Ad35 knob domains: i) Surface plasmon resonance (SPR) analysis was conducted, which allowed measurement of the stringency of specific protein-protein interactions in a highly sensitive manner (Naimushin et al., Biosens. Bioelectron. 17:573-84 (2002)). Efficient binding of recombinant Ad5 and Ad35 fiber knobs to FIX, immobilized on the sensor surface, was readily detected (FIG. 4c). ii) Protein cross-linking assays were conducted with recombinant FIX and increasing amounts of purified recombinant Ad5 or Ad35 knob domains, followed by Western blotting with polyclonal anti-FIX antibodies. This study confirmed the formation of a specific complex between Ad fiber knobs and FIX. iii) In infection competition assay, the addition of purified Ad5 or Ad35 fiber knobs to Ad5F* and FIX-containing media efficiently competed virus infection on HepG2 cells (p<0.01) (FIG. 4d). Based on these data, it was concluded that human coagulation FIX can bind to Ad fiber knob domain and confer efficient infection in a CAR-independent maimer.

To further delineate the role of HSPG and LRP as receptors in FIX-mediated Ad infection, rodent cell lines were utilized where either the LRP locus was knocked out (MEF-Lrp^{-/-}) or glycosylation of HSPG was absent due to a lack of xylosyltransferase (CHO-pgsA745) (Reblin et al., J. Lipid Res. 38:2103-10 (1997); Ji et al., J. Biol. Chem. 268:10160-67 (1993)). Infection of MEF-Lrp^{-/-} cells as well as corresponding MEF-Lrp^{+/+} cells with Ad5F* and Ad5/35L resulted in very poor transduction levels (FIG. 4e) and addition of FIX did not significantly increase infectivity of the Ad vectors. These data suggest that the presence or absence of LRP does not represent a limiting step for virus infection. In contrast, infection of the HSPG-expressing CHO-K1 cells with Ad5F* and Ad5/35L in the presence of FIX resulted in a more than 45-fold increase in reporter gene activities compared to infection without FIX (P<0.001) (FIG. 4f). The effect of FIX was nearly abolished in CHO cells that do not express HSPG (CHO-pgsA745). Taken together, the data demonstrate that HSPG play a major role in mediating the initial binding of the Ad-FIX complex to liver cells in vitro and in vivo.

To test whether this new mechanism of Ad infection could be applicable to human cells, primary human hepatocytes were infected with Ad5L1 and Ad5F* vectors in the presence or absence of human FIX. The CAR-binding ablated Ad5F* vector infected primary human hepatocytes very poorly. Addition of FIX increased the level of Ad5F* transduction by more than 14 fold (P=0.0033). FIX-mediated Ad5F* infection of human hepatocytes was significantly inhibited by lactoferrin and nearly completely abolished by heparin, which blocks the receptor recognition epitopes on activated FIX (Neels et al., Blood 96:3459-65 (2000)).

TFPI is also involved in Ad5 liver transduction. Tissue factor pathway inhibitor protein (TFPI) supports binds to the same class of receptors on liver cells as FIX. The addition of TFPI to Ad5F*, CAR-binding ablated Ad5-based vector allows for 30 to 50 times more efficient transduction of CHO cells, compared to saline added control infections. The TFPI-mediated infection of CHO cells can be efficiently competed by recombinant purified Ad5 or Ad35 fiber knob domains, demonstrating that TFPI-mediated infection requires direct interaction of blood factors with the fiber knob domain.

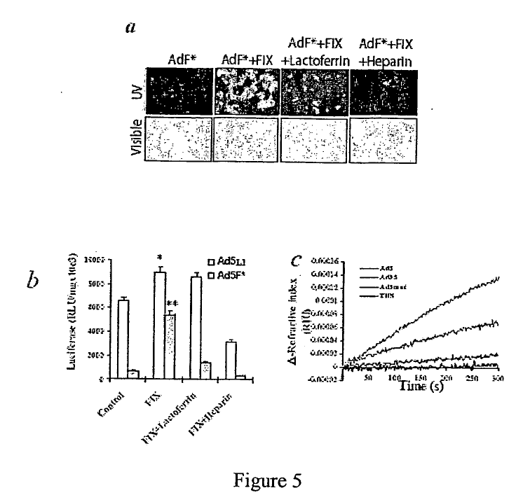

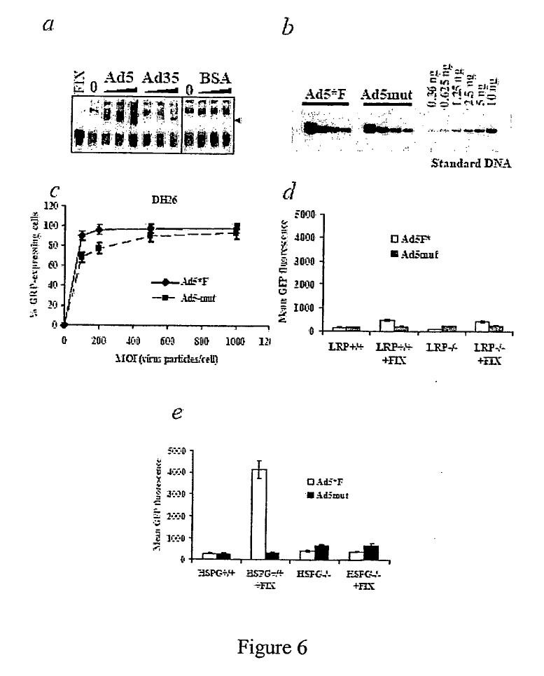

A mutant Ad vector ablated for binding to CAR and FIX does not efficiently transduce liver cells in vivo. To confirm the involvement of FIX in liver transduction, an Ad vector was generated that is incapable of binding to FIX. Based on molecular modeling of fiber knob, specific mutations were introduced into the Ad5F* fiber knob domain. After screening of a number of mutants, the following modification were found to significantly reduce binding to FIX: i) The Y477A single point mutation ablating Ad binding to CAR was combined with an FG-loop deletion (ΔTAYT) that was predicted to change the overall conformation of the knob without disturbing its ability to trimerize. ii) The HI-loop was extended by inserting a 12 amino acid long heterologous peptide (position 547) to create additional sterical hindrances preventing the interaction with natural ligands. The C-terminus of the mutant fiber knob also contained a six histidine tag that allowed for the purification of recombinant knob and for propagation of a corresponding virus. The recombinant knob domain possessing these mutations (Ad5mut) was expressed in E. coli. SPR analysis demonstrated that the Ad5mut fiber knob domain binds significantly less efficiently to FIX, compared to unmodified Ad5 or Ad35 knobs (FIG. 5c). An Ad vector containing the mutated knob was rescued and propagated to the high titers on 293-DH26 cells. The fact that Ad5mut efficiently infects 293-DH26 cells (FIGS. 6b and c) demonstrates that this virus is viable and can use the artificial receptor to gain cell entry. At the same time, Ad5mut was unable to efficiently infect cells in vitro that did not express the artificial receptor and addition of FIX did not restore its infectivity, in contrast to Ad5F* (FIGS. 6d and e).

To evaluate the ability of Ad5mut virus to infect liver cells in vivo, 10¹¹ particles of Ad5L1, Ad5/35L-GFP and Ad5mut vectors were injected intravenously into mice. Quantitative Southern blot analysis for vector genomes performed 72 hours post-infusion, demonstrated that, compared to Ad5L1 and Ad5/35L, about 50 times less Ad5mut vector was present in the liver. Analysis of GFP expression on liver section corroborated the data obtained by the Southern blot analysis. While more than 95% of hepatocytes stained positive for GFP in mice injected with Ad5L1 or Ad5/35L-GFP, only sparse transduced hepatocytes were found after injection with Ad5mut vector.

To evaluate whether accumulation of Ad in Kupffer cells is also affected by the fiber knob mutations, the distribution of Cy-3 labeled Ads was analyzed on liver sections 30 minutes after tail vein injection. Ad5L1 and Ad5/35L efficiently accumulated in Kupffer cells (identified by staining for the macrophage-specific marker F4/80). In contrast, Ad5mut, while detectable throughout the liver parenchyma, did not significantly co-localize with F4/80 staining. Notably, isolated liver perfusion (with saline) with Ad5L1 as well as with Ad5mut did not result in a significant accumulation of virus in Kupffer cells. Perfusion of livers with virus and FIX resulted in restoration of Ad5L1 vector infection of Kupffer cells, however the presence of FIX had no effect on Ad5mut tissue distribution.

Taken together, the data demonstrate that for CAR-binding ablated vectors, infection of primary human hepatocytes in vitro and murine hepatocytes and Kupffer cells in vivo is mediated through interaction with FIX. For CAR-interacting vectors, the FIX mediated pathway is the dominant pathway for Ad uptake into Kupffer cells in vivo. Specific mutations introduced into the Ad fiber knob domain ablating both binding to CAR and FIX allow for a significant reduction of Ad accumulation in liver cells after systemic application.

Discussion

In this study, a new pathway is described that is utilized by Ad for infection of liver, which is the organ predominantly transduced after systemic vector application. The data challenge the currently accepted model of Ad infection that is based mostly on in vitro analyses. According to this model, Ad infects cells in a two step process (Wickham et al., Cell 73:309-19 (1993)). The first and limiting step is the binding of Ad fibers to the primary cell surface receptor. Next, RGD motifs within the Ad penton base interact with cellular integrins, allowing for internalization of the attached virus particles into the cell. An important consequence of this model was the generalization that cells which do not efficiently express the primary attachment receptor(s) would be refractory to Ad infection.

Here, the results demonstrated that Ads, which were unable to infect hepatocytes in vitro due to an inability to interact with CAR, efficiently infected hepatocytes in vivo. Based on these findings, a new model is presented in which in vivo infection of hepatic cells by Ads occurs through binding of viral particles to blood factors, in particular to coagulation FIX, and re-directing these complexes to hepatocellular receptors, including HSPGs. These data demonstrate that Ad vectors containing the Ad5 fiber shaft (Ad5/35L and especially Ad5F*, possessing only a single point mutation within an exposed loop of Ad knob) are unable to infect hepatocytes in vivo in the absence of blood factors, arguing against a major role of the fiber shaft motif in Ad infection in vivo. Furthermore, multiple lines of evidence indicate that the Ad fiber knob domain is the major determinant within the Ad capsid responsible for liver infection in vivo. i) The in vivo infectivity of short-shafted Ad vectors, which differed only in their fiber knob domains varied over a 20-fold range (Ad5S and Ad5/9S). ii) SPR analysis and protein cross-linking assays demonstrated direct binding of recombinant Ad knob domains to FIX. (FIG. 4c). iii) Purified recombinant Ad knobs can compete for FIX-mediated Ad5F* infection (FIG. 4d), and iv) The introduction of mutations in the fiber knob domain reduced FIX-binding in vitro and liver infection in vivo (FIG. 5 and FIG. 6c-e). Although these results show the FIX-mediated pathway is sufficient to provide CAR-independent Ad infection of liver cells in vivo, other blood factors or their complexes may also be involved in this process. In this context it is notable that administration of Ad5/35L vector in FIX-knock-out mice resulted in substantial hepatocyte transduction in vivo. TFPI supports infection of hepatocyte with non-CAR interacting vectors in vitro, albeit only at 50 fold higher than physiological concentrations. Importantly, the specific mutations introduced into the fiber knob domain were sufficient to prevent Ad accumulation in Kupffer cells and transduction of hepatocytes in vivo.

These studies with Ads containing modified fibers possessing Ad5, Ad9, or Ad35 (subgroup C, D, and B, respectively) knob domains suggest that this new pathway can be utilized by different Ad serotypes. A crucial role of virus interaction with blood factors in viral pathogenesis was suggested for several members of the Flaviviridae family, including hepatitis C and dengue viruses (Agnello et al., Proc. Natl. Acad. Sci. USA 96:12766-71 (1999); Hilgard et al., Hepatology 32:1069-77 (2000)). Adenoviruses are a new addition to the list of viruses that use an indirect mechanism of target cell infection. Thus, it is apparent that Ads use multiple pathways for infection in vivo, which may represent an evolutionary mechanism to extend the spectrum of target cells.

The Ad uptake by Kupffer cells in vivo does not involve the CAR-pathway but is mediated by FIX. The FIX-mediated pathway may be dominant in the presence of anti-Ad antibodies, which could mediate Ad uptake via Fc receptors present on Kupffer cells. Kupffer cells are responsible for eliminating the vast majority of intravenously injected Ad and triggering an innate immune response as well as vector toxicity (Worgall et al., Hum. Gene Ther. 8:37-44 (1997); Lieber et al. J. Virol. 71:8798-807 (1997)). In this context, it is notable that the death of a patient in 1999 after systemic Ad vector application resulted from an innate immune response (Raper et al, Hum. Gene Ther. 13:163-75 (2002)). The discovery of the FDC-mediated mechanism for uptake of native as well as CAR-binding ablated Ad5 based vectors by Kupffer cells should be taken into account for the evaluation of safety and efficacy of Ad vector in clinical trials.

These findings have immediate practical implications for re-directing adenovirus tropism to increase gene delivery to target tissues. For re-targeting, the native Ad tropism is abolished and a new binding ligand with specificity for the target tissue is added. Future re-targeting strategies should consider both the interactions between the fiber knob and FIX as well as the interaction with the primary attachment receptor.

### Example 2

In this study, Ad vectors with modified fibers were studied to understand the morphological structures and mechanisms that govern the early accumulation of Ad in the liver.

Methods

Ad vectors. The following Ad vectors, expressing green fluorescent protein (GFP) or β-galactosidase reporter genes, were used: Ad5/9L, Ad5/9S, Ad5/35L, and Ad5/35S (Shayakhmetov et al., J. Virol. 74:10274-86 (2000)). Ad5/9L and Ad5/9S possess the Ad9 fiber knob domain and the long Ad5 fiber shaft (Ad5/9L) or the short Ad9 fiber shaft (Ad5/9S). Ad5/35L and Ad5/35S possess the Ad35 fiber knob domain and the long Ad5 fiber shaft (Ad5/35L) or the short Ad35 fiber shaft (Ad5/35S). For comparative analyses, identical GFP (for Ad5/9 vectors) and β-galactosidase (for Ad5/35 vectors) reporter gene expression cassettes were introduced into the E3 region of the Ad genome by homologous recombination in Escherichia coli strain BJ as described earlier (Shayakhmetov et al., J. Virol. 74:2567-83 (2000)). For amplification, all Ads were used to infect 293 cells under conditions that prevented cross-contamination. Viruses were banded in CsCl gradients, dialyzed, and stored in aliquots as described elsewhere (Carlson et al., Methods Enzymol. 346:277-92 (2002)). Ad genome titers were determined by quantitative Southern blotting. Virion DNA extracted from purified virus particles for each Ad vector was run on an agarose gel in serial two-fold dilutions. As standard DNA of a known concentration (determined spectrophotometrically), preparatively purified Ad5 DNA was used. Standard DNA was applied to the same gel in serial dilutions. After transfer to Hybond N+ nylon membranes (Amersham, Piscataway, N.J.), filters were hybridized with a labeled DNA probe (8-kb HindIII fragment, corresponding to the E2 region of the Ad5 genome), and DNA concentrations were measured with a PhosphorImager (Molecular Dynamics, Sunnyvale, Calif.) for each virus preparation. These values were used to calculate the genome titer for each virus stock used. For each Ad vector used in this study, at least two independently prepared virus stocks were obtained and characterized by determination of PFU titers on 293 cells (Mittereder et al., J. Virol. 70:7498-509 (1996)) and determination of genome titers by Southern blotting (Shayakhmetov et al., J. Virol. 74:2567-83 (2000)). The genome/PFU ratios were comparable for all vectors used in this study.

Each produced virus stock was tested for endotoxin contamination by using Limulus amebocyte lysate Pyrotell (Cape Cod Inc., Falmouth, Mass.). For in vivo studies, only virus preparations confirmed to be free of endotoxin contamination were used.

Ad infection in vivo. All study procedures involving animals were conducted in accordance with the institutional guidelines set forth by the University of Washington. C57BL/6 mice (Charles River, Wilmington, Mass.) were housed in specific-pathogen-free facilities. For analysis of Ad-mediated gene transfer into liver cells, 10¹¹ Ad genomes or particles (corresponding to 5×10⁹ PFU of Ad5/35L vector, determined on 293 cells) in 200 μl of phosphate buffered saline (PBS) were injected by tail vein infusion. For in vivo transduction studies, mice were sacrificed at 72 hours after virus infusion and livers were processed for histological analysis. For analysis of Ad genome accumulation in liver tissue, at 30 minutes, 6 hours, and 24 hours after Ad vector administration into the tail vein, the blood was flushed from the liver with a cardiac saline perfusion, the liver was harvested, and total DNA was purified as described earlier (Shayakhmetov et al., Cancer Res. 62:1063-68 (2002)).

To analyze Ad association with purified hepatocytes, at 30 minutes after tail vein vector application, mouse livers were perfused for 15 minutes with a collagenase solution via a catheter permanently placed into the portal vein (Vrancken Peeters et al., BioTechniques 20:278-85 (1996)). Then, partially disintegrated livers were carefully removed and dispersed in a collagenase-DNase solution to a single-cell suspension. Following two consecutive washes with 40 ml of P13S containing 2% fetal bovine serum and two differential centrifugations (500×g for 5 minutes each time), allowing efficient sedimentation of only hepatic parenchymal cells, purified hepatocytes were obtained. Routinely, the purity of hepatic cells obtained by this technique was greater than 90%, according to anti-albumin immunochemical staining. For further analyses, the purified hepatocytes either were immediately lysed with pronase-sodium dodecyl sulfate buffer for obtaining total cellular DNA or were plated on six-well Primaria plates and cultured for 24 to 48 hours. To analyze virus degradation in purified hepatocytes over time, hepatocytes were purified by collagenase perfusion only. At 24 hours after plating, hepatocytes were treated with a collagenase-DNase or trypsin-DNase solution for 15 minutes at 37° C. to remove all extracellular virus, washed with PBS, and lysed with pronase-sodium dodecyl sulfate buffer to obtain cellular DNA for subsequent Southern blot analysis. To analyze the efficiency of Ad vector interactions with hepatic cells, livers were perfused for 15 minutes with 0.25% trypsin-DNase solution prior to collagenase perfusion, and hepatocytes were isolated as described above.

For analysis of reporter gene expression in hepatocytes, at 48 hours after purification and plating, photographs of cells were taken under visible light and UV light (for analysis of GFP expression) or cells were fixed and stained in situ for β-galactosidase activity as described earlier (Shayakhmetov et al., J. Virol. 74: 10274-86 (2000)).

Analysis of Ad-Kupffer cell interactions in vivo. To analyze Ad interactions with Kupffer cells, the Ad vectors were labeled with the fluorophore Cy-3 (Shayakhmetov et al., J. Virol. 77:3712-23 (2003)). The titers of Cy-3-labeled vectors were confirmed by quantitative Southern blotting. A total of 10¹¹ particles of fluorophore-labeled Ads were injected into the tail vein; 30 minutes later, livers were flushed with saline via cardiac perfusion, harvested, and immediately frozen in OCT compound. Frozen liver sections either remained unstained or were stained with rat anti-mouse F4/80 or CD45 primary antibody (BD Biosciences, Palo Alto, Calif.) to detect Kupffer cells. Specific binding of primary antibodies was visualized with secondary anti-rat Alexa Fluor 488 antibody (green) (Molecular Probes, Inc., Eugene, Oreg.). Cy-3-labeled Ad particles appear red. When gadolinium chloride (GdCl3) was used to deplete mice of Kupffer cells, two doses of the drug were injected into mice at 30 hours and 6 hours before Ad administration as described (Lieber et al., J. Virol. 71:8798-807 (1997)).

Southern blot analysis. Isolation of cellular DNA from mouse livers and Southern analysis were performed as described elsewhere (Lieber et al., J. Virol. 71:8798-807 (1997)). A ³²P-labeled 8-kb HindIII fragment, corresponding to the E2 region of the Ad genome, was used for hybridization to specifically detect Ad genomic DNA in livers and purified hepatocytes.

Electron microscopy analysis. For electron microscopy analysis of virus distribution in liver tissue, 10¹¹ Ad particles in a total volume of 200 μl of PBS were injected into the tail vein. At 30 minutes after virus application, livers were harvested, fixed with 2% glutaraldehyde in PBS, and subsequently fixed in 1% OsO₄-phosphate buffer. Then, liver samples were embedded in Medcast (Ted Pella, Redding, Calif.), and ultrathin sections were stained with uranyl acetate and lead citrate. Processed grids were evaluated with a Philips 410 electron microscope operated at 80 kV (magnification, ×21,000). At least two mice were injected with each Ad vector, and the intracellular distribution was analyzed by using multiple liver sections.

Analysis of levels of cytokines, chemokines, and aminotransferases in plasma. To analyze the levels of proinflammatory cytokines and chemokines in serum, at 30 minutes, 6 hours, and 24 hours after intravenous administration of Ad vectors, blood samples were collected in heparin-treated Eppendorf tubes, cells were pelleted for 5 minutes at 1,000×g, and plasma was obtained and stored at −80° C. in small aliquots. To analyze the levels of cytokines and chemokines in plasma, a mouse inflammatory cytometric bead array (BD Biosciences) was used. Briefly, 10 μl of mouse plasma was diluted five times and mixed with cytometric beads capable of binding mouse TNF-α, IL-6, monocyte chemoattractant protein 1, IFN-γ,IL-12p70, and IL-10. The binding of these proteins was detected with corresponding secondary phycoerythrin-conjugated antibodies and analyzed by flow cytometry along with provided standard proteins. The collected data were processed by using the manufacturer's software. Plasma samples obtained from at least three mice (for each Ad vector) were analyzed in duplicate. To analyze the levels of alanine aminotransferase (ALT), a marker of hepatocellular damage, in plasma, a calorimetric ALT detection protocol and reagents (TECO Diagnostics, Anaheim, Calif.) were used according to the manufacturer's protocol without modifications. ALT levels were measured in triplicate by using plasma samples obtained from at least three mice (per Ad).

RNase protection assay. To analyze the mRNA levels for multiple cytokine and chemokine genes in mouse livers, 10¹¹ Ad particles were injected into the tail vein; at 30 minutes, 6 hours, and 24 hours after virus injection, livers were harvested, and total RNA was extracted by using an RNAqueous-midi kit (Ambion, Inc., Austin, Tex.). Ten μg of total liver RNA was hybridized with a mixture of ³²P-labeled RNA probes. The ³²P-labeled RNA probe mixture was prepared by in vitro transcription with an in vitro transcription kit and CK-3 and a custom template set provided by Pharmingen (San Diego, Calif.). The hybridized RNA was treated with RNase by using an RNase protection assay kit (Pharmingen) and precipitated, and the protected fragments were resolved on vertical sequencing (10% acrylamide) gels. Following electrophoresis, the gels were dried and exposed to X-ray film (Kodak-X-Omat) and a PhosphorImager screen. The signals on the screen were analyzed with PhosphorImager software. The RNase protection assay was performed with RNA samples from two to five individual livers (for each virus). At least two independently prepared virus stocks were used for this analysis.

Results

Immediately after injection, all Ad vectors accumulated with equal efficiencies in the liver, but only genomes of long-shafted Ads are efficiently maintained over time. To delineate the mechanisms involved in Ad accumulation in the mouse liver following intravenous application, a set of Ad5-based vectors possessing modified fibers was used (Shayakhmetov et al., J. Virol. 74:10274-86 (2000)). Ad5/9L and Ad5/9S possess the Ad9 knob domain and long (Ad5, 22 β-sheets) or short (Ad9, 7 β sheets) fiber shafts, respectively. Ad5/35L and Ad5/35S possess long (Ad5) and short (Ad35) fiber shafts and an Ad35 knob domain. Adenovirus vectors possessing the Ad9 knob domain can infect cells in vitro via interactions with CAR (Roelvink et al., J. Virol. 72:7909-15 (1998); Shayakhmetov et al., J. Virol. 74:10274-86 (2000)), while Ad35 fiber knob-possessing vectors can infect cells via interactions with CD46 (Gaggar et al., Nat. Med. 9:1408-12 (2003)). As outlined above, rodent cells in vitro do not express CD46 and are refractory to infection with Ad35 fiber knob-possessing vectors. In vivo, long-shafted Ad35 knob-possessing vectors (Ad5/35L) can infect hepatocytes as efficiently as Ad5 vectors. This infection is presumably mediated by blood factors and uptake through HSPGs and low-density lipoprotein receptor-related protein (Shayakhmetov et al., Mol. Ther. 7:S16539 (2003)). Previous data suggested that this pathway cannot be used efficiently by short-shafted vectors, since the accumulation of Ad genomes and transgene expression in hepatocytes for short-shafted Ad5/35S was 1/10 that of Ad5 vectors when measured 72 hours after tail vein injection (Bernt et al., Mol. Ther. 8:746-55 (2003); Shayakhmetov et al., Cancer Res. 62:1063-68 (2002)).

To analyze whether the reduced efficiency of transgene expression seen for the short-shafted non-CAR-interacting Ad5/35S vector would also be observed for the short-shafted CAR-interacting Ad5/9S variant, 10¹¹ particles of each Ad were administered into the tail vein of C57BL/6 mice. Three days later, mice were sacrificed, livers were fixed in formalin, and serial sections were prepared. The analysis of GFP expression revealed that only long-shafted Ads were able to efficiently transduce hepatocytes and express the transgene by this time, regardless of the type of fiber knob domain.

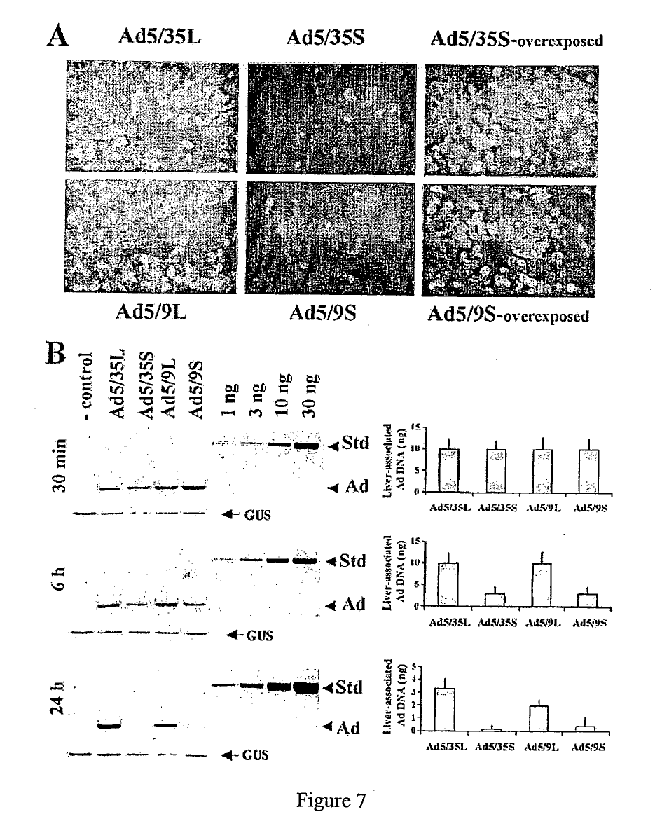

To analyze the kinetics of Ad accumulation in the liver at earlier time points, 10¹¹ particles of each viral vector were administered into the tail vein of C57BL/6 mice. At 30 minutes, 6 hours, and 24 hours post-infusion (p.i.), blood was flushed from the liver by cardiac saline perfusion, livers were harvested, and hepatic DNA was subjected to quantitative Southern blot analysis with a probe specific to viral DNA. At 30 minutes p.i., the amounts of Ad DNA in the liver were comparable for all vectors. However, at 6 hours p.i., and more so at 24 hours p.i., there was less viral DNA of short-shafted vectors in the liver than of the corresponding long-shafted variants. In agreement with earlier findings (Worgall et al., Hum. Gene Ther. 8:37-44 (1997)), the total amount of long-shafted Ad DNA in the liver declined by more than 80% within 24 hours p.i. (FIG. 7; compare the intensity of the viral bands to that of the standard at 30 minutes and 24 hours). In conclusion, immediately after injection, regardless of the shaft length, viral particles are deposited equally efficiently in the liver. However, only long-shafted Ad genomes are efficiently retained in the liver over time.

At 30 minutes, short- and long-shafted Ad5/9 and Ad5/35 particles are localized to the liver sinusoids. To analyze in more detail the localization of Ad particles within the liver at 30 minutes, electron microscopy was performed on ultrathin liver sections. Virus particles of both long-shafted and short-shafted vectors could be found in the sinusoids and the space of Disse, suggesting that all Ads are physically capable of reaching the hepatocyte surface.

Ad9 knob-possessing vectors can infect hepatocytes through CAR, but internalization for Ad5/9S is slower than that for Ad5/9L. To further corroborate the electron microscopy findings, it was determined whether Ad particles were inside or outside liver cells within the first 30 minutes p.i. In these studies, the binding to and uptake by hepatocytes of Ad particles was examined. At 30 minutes after virus administration into the tail vein, the liver was perfused for 15 minutes with a trypsin-DNase solution to disrupt virus-receptor complexes and degrade non-internalized viral DNA. Hepatocytes were isolated by collagenase digestion of liver samples (see Methods). Total DNA isolated from purified hepatocytes was analyzed by quantitative Southern blotting. In hepatocytes obtained by collagenase perfusion (without prior trypsin digestion), the amounts of the Ad5/9L and Ad5/9S vectors were comparable. This finding was unexpected, considering that the infection of cultured cells in vitro was inefficient with short-shafted Ad5/9S due to electrostatic repulsion between the negatively charged particle and the cell surface (Shayakhmetov et al., J. Virol. 74:10274-86 (2000)). It is possible that collagenase perfusion of the liver removes some negative charge from the surface of hepatocytes, facilitating infection with Ad5/9S through CAR. When the liver was perfused with trypsin before hepatocyte isolation, the amount of the short-shafted Ad5/9S vector associated with liver cells was significantly smaller than that of the long-shafted Ad5/9L vector. This finding suggests that trypsin, but not collagenase, treatment disrupted Ad5/9S-CAR complexes (and/or digested viral particles). Furthermore, the data indicate that although the initial attachment of both Ad9 knob-possessing vectors occurred with similar efficiencies, Ad5/9S required more time for internalization into cells.

To analyze whether the fates of Ad particles with different fiber shaft lengths internalized within infected hepatocytes differ, at 30 minutes after Ad infusion, hepatocytes were isolated by collagenase perfusion, plated on six-well plates, and cultured for 24 hours. One set of cells was treated with a collagenase-DNase solution and another set was treated with a trypsin-DNase solution to remove all extracellular virus. Southern blot analysis of hepatocellular DNA showed similar amounts of Ad5/9L and Ad5/9S vector DNAs, indicating that differential intracellular degradation or retrograde transport (Shayakhmetov et al., J. Virol 77:3712-23 (2003)) to the cell surface of Ad5/9S particles, compared to Ad5/9L particles, is not the primary mechanism for the removal of short-shafted vectors from liver cells by 24 hours p.i. in these studies. The presence of the same amounts of Ad5/9L and Ad5/9S genomes in hepatocytes is distinct from the transgene expression data shown in FIG. 7A. Collagenase perfusion of livers (performed to obtain hepatocytes) allows for the binding of Ad5/9S to CAR and for subsequent uptake by hepatocytes. On the other hand, the transgene expression data were obtained at day 3 p.i. without collagenase treatment, a situation that might make CAR accessible to Ad5/9S binding.

Ad5/35S is unable to interact with hepatocellular receptors and is therefore not taken up by hepatocytes. A similar analysis was performed with Ad35 knob-possessing vectors. First, the presence of viral DNA was analyzed immediately after collagenase perfusion. As was seen with the Ad5/9L vector, collagenase perfusion did not affect the amount of the Ad5/35L vector, indicating that collagenase treatment did not interfere with Ad35 fiber knob binding to hepatocellular receptors. However, in contrast to the results obtained for Ad5/9, collagenase perfusion reduced the amount of short-shafted Ad5/35S associated with purified hepatocytes by more than 30-fold. This finding is not unexpected because, compared to Ad5/9S, Ads/35S cannot interact with CAR and can be washed out from the liver. Preperfusion of the liver with a trypsin-DNase solution prior to hepatocyte purification further reduced the amount of Ad5/35S vector DNA associated with purified hepatocytes. Since only a minimal amount of Ad5/35S vector genomes was associated with liver cells at 30 minutes p.i., the amount of Ad5/35S vector genomes associated with purified hepatocytes at 24 hours p.i. was barely detectable. Taken together, these data demonstrate that although the amount of Ad5/35S genomes in the liver tissue was similar to that of Ad5/35L genomes, the short-shafted virus was unable to efficiently interact with cellular receptors for attachment and internalization and therefore was not associated with purified cells.

To further confirm that Ad particles can be internalized during the first 30 minutes p.i., hepatocytes were purified 15 and 30 minutes after Ad infusion. These cells were plated on six-well plates, and reporter gene expression (GFP expression for Ad5/9 vectors and β-galactosidase expression for Ad5/35 vectors) was analyzed 48 hours later. For Ad5/9S and Ad5/9L, efficient hepatocyte transduction occurred within 15 minutes p.i. For Ad5/35L, 60% of cells purified at 15 minutes p.i. and more than 90% of cells purified at 30 minutes p.i. were positive for transgene expression. In contrast, only a few hepatocytes demonstrated transgene expression when Ad5/35S was infused into mice, corroborating the Southern blot data that the genomes of short-shafted Ad5/35S, although present in the liver tissue at an amount similar to that of their long-shafted counterparts, are unable to attach to and productively infect liver cells. Taken together, these results indicate that the accumulation of Ad in liver tissue shortly after intravenous administration does not depend on the interaction of viruses with liver cell receptors.

Ad5/35L is efficiently taken up by Kupffer cells via a CAR-independent, knob-dependent pathway. To investigate in more detail the distribution of Ad vectors in the liver tissue shortly after intravenous application, Ad particles were labeled with the fluorophore Cy-3 (Leopold et al., Hum. Gene Ther. 9:367-78 (1998)). At 30 minutes after infusion of Cy-3-labeled Ads, blood was flushed from the livers, and the livers were harvested and cryosectioned. While the long-shafted Ad5/35L vector formed large virus aggregates, the signals from the short-shafted Ad5/9S and Ad5/35S vectors and the longshafted Ad5/9L vector were smaller, dim, and evenly dispersed throughout the sections. Analysis of liver sections with anti-F4/80 antibodies, which specifically stained macrophages and Kupffer cells (Liu et al., Hum. Gene Ther. 14:62743 (2003)), revealed that the areas of Ad5/35L accumulation overlapped the areas of positive anti-F4/80staining, demonstrating that a significant amount of the longshafted Ad5/35L vector is trapped by Kupffer cells at this time. Although the colocalization of Ad and F4/80 signals was also seen for the other Ads, Kupffer cell uptake of these vectors was significantly less efficient than that of Ad5/35L. Because the long-shafted Ad5/35L and Ad5/9L vectors differ only in their fiber knob domain, the data demonstrate that the Ad fiber knob is the primary determinant within the Ad capsid responsible for Ad accumulation in Kupffer cells. Furthermore, because Ad5/35L does not interact with CAR, Kupffer cell uptake by this vector is CAR independent.

Independently of the fiber length or the nature of the knob domain, Kupffer cells are not the major reservoir in the liver for systemically-applied Ad. To understand the role of Kupffer cells in the fate of Ad5/35L, Kupffer cells were functionally inactivated by gadolinium chloride (GdCl₃) administration into mice, and the levels of Ad accumulation in the livers of normal mice and GdCl₃-treated mice were compared. The administration of GdCl₃ 30 minutes and 6 hours prior to virus injection dramatically reduced the ability of Kupffer cells to accumulate long-shafted Cy-3-labeled Ad5/35L. Nonetheless, quantitative Southern blot analysis of Ad genomes accumulated in the liver tissue at 30 minutes p.i. demonstrated similar levels of Ad DNA in both normal and GdCl₃-treated mice for the long-shafted Ad5/9L and Ad5/35L vectors and the short-shafted Ad5/35S and Ad5/9S vectors.

In conclusion, from the finding that the accumulation of both long-shafted and short-shafted vectors in the liver occurred at similar levels, one can conclude that Kupffer cells are not the major reservoir for intravenously injected Ad in liver tissue.

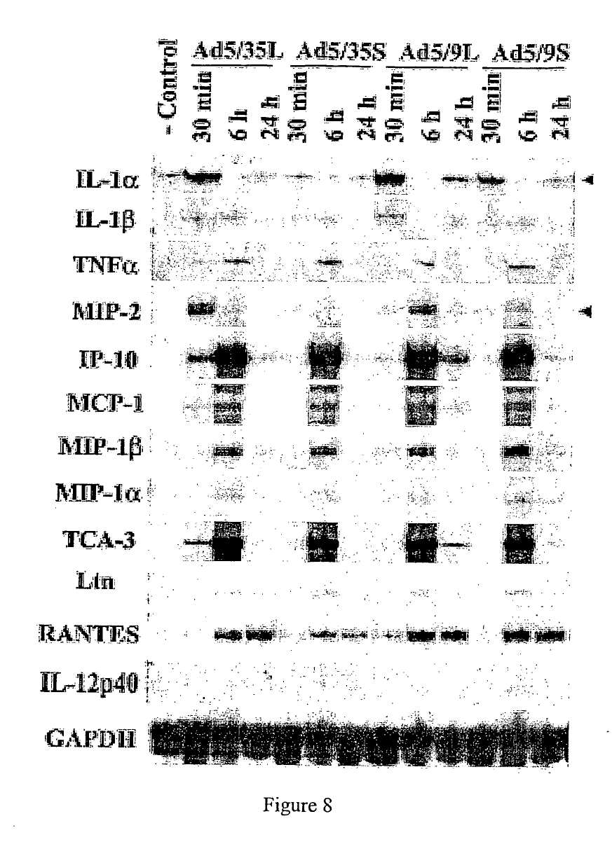

Short-shafted Ads induce less cytokine gene expression in liver cells after systemic application. The intravenous administration of Ad results in the initiation of strong innate immune responses in animals and humans (Raper et al., Mol. Genet. Metab. 80:148-58 (2003)). To evaluate whether efficient liver cell infection is required for the initiation of innate and inflammatory responses, the levels of hepatic cytokine and chemokine gene transcription as well as levels of proinflammatory cytokines and ALT in serum after intravenous Ad administration were analyzed. These analyses revealed that the mRNA levels for most of the genes analyzed at 30 minutes after virus administration were significantly lower for short- shafted vectors than for their long-shafted counterparts (FIG. 8, 30 min). Among all analyzed genes, the transcription levels for the IL-1α, IL-1β, and MIP-2 genes were highest shortly after long-shafted Ad5/35L vector administration. Notably, Ad5/35L was more efficiently taken up by Kupffer cells than the other Ads. In particular, the levels of IL-1α increased by 20-fold and the levels of MIP-2 increased by more than 25-fold for Ad5/35L compared to preinjection levels. Although the upregulation of IL-1α gene transcription in the liver after the administration of the Ad5/9L vector was similar to that seen with the Ad5/35L vector, the levels of MIP-2 mRNA were significantly lower with Ad5/9L vector application than with Ad5/35L vector application. Nonetheless, the majority of the genes analyzed were strongly upregulated after injection of the long-shafted Ad5/9L vector, compared to its short-shafted counterpart. Clearly, the delineation of pathways activated upon Ad uptake requires further investigation.

Importantly for Ad5/35S, the mRNA levels for IL-1α were only slightly elevated and those for MIP-2 were nearly unchanged compared to preinjection levels. Notably, MIP-2 was suggested to be the primary chemokine responsible for the attraction of neutrophils to the liver, an event which subsequently causes liver damage (Liu et al., Hum. Gene Ther. 14:627-43 (2003)). The low levels of MIP-2 chemokine gene expression after the administration of the Ad5/35S and Ad5/9L vectors demonstrate that efficient infection of Kupffer cells most likely is required for its upregulation.

Significant upregulation of IL-10 and TCA-3 chemokine mRNA levels (more than 300-fold above the baseline) at 6 hours p.i. did not depend on the length of the Ad fiber shaft domain or the nature of the fiber knob domain. The delayed upregulation (6 or 24 hours) of these genes in the liver may have more complex mechanisms, potentially involving factors secreted from the spleen, peripheral blood mononuclear cells, and/or cells in the lymph node upon Ad infection, and apparently does not require efficient liver cell transduction.

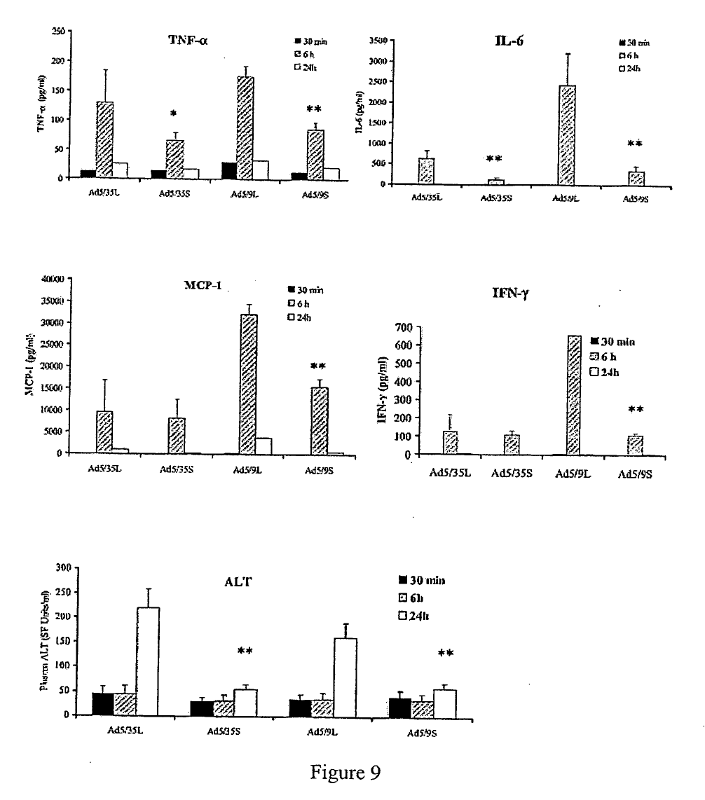

Levels in serum of TNF-α, IL-6, and ALT are higher for long-shafted Ads. Analysis of serum cytokine levels revealed that for all analyzed proteins, peak levels were seen at 6 hours p.i. (FIG. 9). Importantly, the levels of TNF-α and IL-6 were significantly higher for long-shafted vectors than for their short-shafted counterparts. The levels of IFN-γ and monocyte chemoattractant protein 1 were higher for the Ads/9L vector than for its short-shafted counterpart and were not significantly different between long- and short-shafted Ad35 knob-possessing vectors.

The assessment of the overall hepatotoxicity at 24 hours after intravenous virus administration by measurement of serum ALT levels revealed that the highest degree of hepatic injury was observed after injection of long-shafted Ads. The serum ALT levels observed after administration of short-shafted vectors were only slightly elevated above preinjection levels, demonstrating that viruses which are inefficient at infecting liver cells (and potentially cells in other organs) are also unable to induce hepatocellular damage.

Discussion

In this study, Ad vectors with modified fibers were studied to understand the morphological structures and mechanisms that govern the early accumulation of Ad in the liver. The Ad vectors varied in two fiber domains that have been found to determine the specificity and efficacy of Ad infection, the fiber shaft and the fiber knob (Nakamura et al., J. Virol. 77:2512-21 (2003); Roelvink et al., J. Virol. 72:7909-15 (1998); Shayakhmetov et al., J. Virol. 74:10274-86 (2000); Vigne et al., Gene Ther. 10:153-62 (2003); Wu et al., J. Virol. 77:7225-35 (2003)). The fiber knob largely determines Ad tropism. Here, the Ad9 knob, known to bind to CAR, and the Ad35 knob, which binds CD46, were used. Efficient infection in vitro through CAR requires a long, flexible shaft (Shayakhmetov et al., J. Virol. 74:10274-86 (2000); Smith et al., Hum. Gene Ther. 14:777-87 (2003); Vigne et al., Gene Ther. 10:153-62 (2003); Wu et al., J. Virol. 77:7225-35 (2003)), whereas the efficiency of infection through CD46 seems to be independent of the fiber shaft length.

Within 30 minutes after tail vein injection, all vectors, independent of the nature of the fiber knob or shaft, accumulated equally efficiently in the liver sinusoids. However, the amount of viral genomes and the number of transgene-expressing hepatocytes were more than 10-fold lower for the short-shafted vectors (Ad5/9S and Ad5/35S) at 24 hours p.i. than for their longshafted counterparts. These data are consistent with a recent study of long- and short-shafted Ad5/40 vectors by Nakamura et al. (Nakamura et al., J. Virol. 77:2512-21 (2003)). They also found no significant differences in the amounts of viral DNA between CAR-binding and non-CAR-binding Ad vectors in the liver, corroborating the notion that early Ad accumulation in the liver does not depend on an interaction with CAR.

Upon accumulation within the liver sinusoids, long-shafted Ad5/9L and Ad5/35L efficiently infect hepatocytes. Both CAR and non-CAR-interacting vectors are equally efficient in hepatocyte transduction in vivo, indicating that the CAR pathway is not the dominant pathway for in vivo infection. The KKTK shaft motif was recently suggested to mediate liver infection through HSPGs in vivo (Smith et al., Hum. Gene Ther. 14:777-87 (2003)). Shayakhmetov et al. have found an additional pathway which appears to dominate both the CAR and the shaft motif pathways (Shayakhmetov et al., Mol. Ther. 7:S165 (2003)). When a liver is perfused with Ad without blood, only minimal hepatocyte transduction is seen with a KKTK shaft motif-containing, non-CAR-interacting Ad5/35L vector, whereas transduction with a CAR-interacting Ad5L vector is not significantly decreased (Shayakhmetov et al., Mol. Ther. 7:S165 (2003)). The latter finding argues against the hypotheses that CAR is not accessible in the liver in vivo and that direct KKTK shaft motif binding to HSPGs is the primary pathway of liver cell infection in vivo.

Short-shafted Ads are unable to infect liver cells through CAR, through the KKTK shaft motif, or through blood factors. Due to these features, they are not taken up by liver cells and are probably degraded within the sinusoids. Importantly, short-shafted Ads did not cause innate toxicity in mice and therefore might represent a useful scaffold for the insertion of targeting ligands. However, when using short-shafted fibers, one must consider that certain ligand-receptor interactions require long fiber shafts. For example, the CAR-knob interaction is not efficient with short fiber shafts, likely because of either steric hindrances that affect the interaction with CAR and/or integrins or repulsion between negative charges on the virion and the cell surface (Nakamura et al., J. Virol. 77:2512-21 (2003); Shayakhmetov et al., J. Virol. 74:10274-86 (2000); Vigne et al., Gene Ther. 10:153-62 (2003); Wu et al., J. Virol. 77:7225-35 (2003)). Theoretically, receptors for short-shafted Ads should protrude over the glycocalyx or should be internal (without the requirement of binding secondary receptors, such as integrins). A natural receptor for short-shafted group B Ads is CD46 (Gaggar et al., Nat. Med. 9:1408-12 (2003)). CD46 is overexpressed on important target cells for gene therapy, including tumor cells (Hara et al., Br. J. Haematol. 82:368-73 (1992); Kinugasa et al., Br. J. Cancer 80:1820-25 (1999); Murray et al., Gynecol. Oncol. 76:176-82 (2000); Thorsteinsson et al., APMIS 106:869-78 (1998)) and potential hematopoietic stem cells (Cho et al., Clin. Exp. Immunol. 83:257-61 (1991); Manchester et al., J. Virol 76:6636-42 (2002)), and vectors that target CD46 are becoming useful tools for gene therapy. Both chimeric Ad5 vectors with E1 deleted but possessing Ad35 fibers (Gao et al., Gene Ther. 10:1941-49 (2003); Mizuguchi et al., Gene 285:69-77 (2002); Reddy et al, Virology 311:384-93 (2003); Sakurai et al., Mol. Ther. 8:813-21 (2003); Shayakhmetov et al, Cancer Res. 62:1063-68 (2002); Shayakhmetov et al., J. Virol. 74:2567-83 (2000); Vogels et al., J. Virol. 77:8263-71 (2003)) and vectors completely derived from Ad35 have been developed, and the generation of empty Ad35 or other group B Ad vectors is in progress (Stone et al., Mol. Ther. 7:S192 (2003)). As shown in this study, in addition to the useful tropism, Ad35 fiber-possessing Ad vectors avoid liver sequestration and innate toxicity. Furthermore, there is a low serum prevalence of neutralizing antibodies against Ad35 in healthy individuals in different parts of the world (Vogels J. Virol. 77:8263-71 (2003)).

To further confirm that Ad35 fiber-containing vectors are safer than Ad5 vectors, studies with transgenic mice that express CD46 in a pattern that mimics that in humans (Kemper et al., Clin. Exp. Immunol. 124:180-89 (2001)) might be conducted. Preliminary data indicate that the amount of Ad5/35 vector DNA in the liver of CD46-transgenic mice is approximately 10 times lower than that of Ad5 vector DNA after systemic vector application (Gaggar et al., Nat. Med. 9:1408-12 (2003)).

Kupffer cells account for 80 to 90% of resident macrophages in the entire body and, after systemic administration, efficiently take up Ad particles. The mechanisms of Ad uptake and subsequent Kupffer cell activation are a matter of intense investigation. This study and others (Liu et al., Hum. Gene Ther. 14:62743 (2003)) have established that Ad uptake by Kupffer cells is CAR-independent. The contribution of the KKTK shaft motif and/or blood factors remains to be shown. Furthermore, in preimmunized animals, Ad-antibody complexes may be taken up by Kupffer cells through Fc receptors. In this context, it is notable that, when high Ad doses were used in preimmunized nonhuman primates, new toxic effects that were not observed in naive animals were found (Varnavski et al., J. Virol. 76:5711-19 (2002)).

As outlined earlier, at doses usually used to achieve efficient transgene expression in target cells (i.e., 2×10⁹ PFU/mouse for the transduction of hepatocytes or tumor cells in tumor models), the Kupffer cell system becomes saturated and virus disseminates and infects other cell types. Furthermore, these data and data obtained by Liu et al. (Liu et al., Hum. Gene Ther. 14:627-43 (2003)) demonstrate that in Kupffer cell-depleted animals, the amount of viral DNA found in the liver early after injection (30 min) is not significantly decreased, indicating that although Kupffer cells efficiently take up Ad, they are not a major reservoir of Ad in the liver. It seems that the majority of virus remains in the liver sinusoids. Schiedner et al. (Hum. Gene Ther. 14:1631-41 (2003)) suggested recently that the activation of Kupffer cells within minutes after Ad injection results in the release of factors which, in turn, activate endothelial cells. These events are associated with thickening of the endothelium, presumably leading to a change in fenestration size. One could speculate that this effect might cause a blockage of trans-endothelial Ad transport, resulting in Ad particles being retained inside the sinusoids or the space of Disse. This situation would subject vector particles to increased clearance from the liver by the bloodstream and circulating innate effector cells (Sakurai et al., Mol. Ther. 8:813-21 (2003)). Arguing against this model is the recent finding that although short-shafted Ad5/35 vectors did not infect mouse tissue and were not taken up by Kupffer cells, they did not transduce liver metastases derived from CD46-positive human tumor cells more efficiently than non-targeted Ad5 vectors (Bernt et al., Mol. Ther. 8:746-55 (2003)). This finding indicates that the mechanisms that govern transendothelial transport of Ad particles are more complex. For example, the shafts of Ad5/35S (which lacks a shaft domain that confers flexibility in Ad5) might not be flexible enough to pass through the endothelial fenestrae. Furthermore, it is possible that Ad, while inside the sinusoids, must contact its cellular receptors through the fiber and is then actively pulled through the fenestrae. Apparently, Ad transendothelial transport is facilitated by the long fiber shaft.

First-generation Ads induced a biphasic pattern of inflammatory cytokine gene expression in the liver after systemic Ad injection. A first peak is seen at about 6 hours p.i. and is associated with virus uptake. A second peak appears at day 4 or 5 p.i. and is associated with viral gene expression (Lieber et al., J. Virol. 71:8798-807 (1997)). Considering the relatively low capacity of Kupffer cells to take up Ad, the question arises as to what other cytokine-producing cell types interact with Ad. Studies by Lui et al. (Hum. Gene Ther. 14:627-43 (2003)) showed that RGD mutants induced less E-selectin and VCAM-1 gene expression in the liver, indicating that Ad infects endothelial cells through integrins, which lead to their activation. Furthermore, peripheral blood leukocytes that infiltrate the liver upon Ad injection may interact with Ad vectors, resulting in cytokine expression. The induction of IL-6, granulocyte-macrophage colony-stimulating factor, and a panel of chemokines upon Ad interaction with peripheral blood cells has been reported (Higginbotham et al., Hum. Gene Ther. 13:129-41 (2002)). Clearly, in situ hybridization or in situ reverse transcription-PCR for cytokine mRNA in liver sections will help to delineate the relative contributions of different cell types in hepatic cytokine expression.

The finding that short-shafted Ads do not cause cytokine expression and release or hepatotoxicity provides a rationale to use these vectors for targeting purposes. We have also shown that short-shafted Ads are a useful tool for analyzing the mechanisms involved in Ad liver sequestration.

### Example 3

In this example, blood factors interacting with an Adenovirus capsid protein *fiber knob domains) are isolated.

Methods

Whole fresh plasma was collected and mixed with Ni-agarose beads (Qiagen Inc, Calif.) and incubated at 4° C. for 1 hour. Next, the beads were removed by centrifugation. Pre-cleared plasma is mixed with Ni-agarose beads covered with purified recombinant Ad5, Ad35 or fiber knob domain of other adenovirus serotypes. Following a subsequent incubation for 1 hour at 4° C., the plasma was discarded, the beads washed 3 to 5 times with phosphate buffered saline, and plasma proteins bound to fiber knob domains are recovered by elution with 8 M Urea. The eluted proteins were dialyzed and then subjected to protein gel analysis, and/or directly processed for mass spectrometry analyses.

Results

Blood factor proteins that bound to Ad5 or Ad35 were recovered. The plasma proteins interacting with these Ad fiber knob domains in EDTA preserved plasma were identified by mass spectrometry analysis. These proteins were pregnancy zone protein, C4, Hemopexin, C4BP, Es-1 (elastase-1), Fibrinogen, Alpha-1-Proteinase Inhibitor and Coagulation factor IX. Binding of C4BP, Fibrinogen and Coagulation factor IX to the fiber knob domains was further confirmed by additional assays.

In vitro analyses of plasma protein interaction with Ad fiber knob was confirmed. A human recombinant C4BP protein, human fibrinogen, mouse fibrinogen, human recombinant CD46, Ad35 fiber knob domain and bovine serum albumin (10 μg each) were loaded onto Hybond-C membrane, and incubated with purified recombinant biotin-conjugated Ad35 fiber knob domain. The specific binding of Ad35 knob domain was developed using peroxidase-conjugated streptavidin. C4BP, mouse fibrinogen and CD46 (the natural receptor for Ad35) demonstrated specific binding to Ad35 fiber knob domain, thus confirming data obtained from mass spectrometry analyses.

In a second confirmatory assay, competition of Ad5/35 virus infection of CHO-C2 cells with human recombinant C4BP protein was performed. The physiological plasma C4BP concentration is 150 μg/ml. Increasing concentrations of C4BP protein, at 25, 50 and 100 μg/ml decreased Ad5.35 infection, as compared with a control lacking C4BP.

The examples and embodiments described herein are for illustrative purposes only and various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An adenovirus fiber comprising a binding site for a cellular receptor, the adenovirus fiber comprising a mutation of a residue of the fiber characterized in that the residue is directed to the binding site for a blood factor protein, wherein the mutation reduces the affinity of the fiber for the blood factor protein.

2. The adenovirus fiber according to claim 1, wherein the blood factor protein is Factor IX, TFPI, C3-precursor, complement C4, complement C4BP, hemopexin, fibrinogen, elastase-1 or pregnancy zone protein.

3. The adenovirus fiber according to claim 1, characterized in that the mutation is in the fiber knob.

4. The adenovirus fiber according to claim 3, characterized in that the mutation is in the AB, FG or HI loop.

5. The adenovirus fiber according to claim 1, wherein the adenovirus fiber comprises a short shaft.

6. The adenovirus fiber according to claim 1, characterized in that the mutation is in a loop in the fiber knob, wherein the affinity of the fiber knob for the native cellular receptor is not substantially reduced.

7. The adenovirus fiber according to claim 6, characterized in that the fiber knob is derived from a type 5 adenovirus (Ad5) fiber knob.

8. The Ad5 adenovirus fiber according to claim 7, characterized in that it is modified by mutation of a residue in the AB, EF or HI exposed loop.

9. The adenovirus fiber according to claim 6, characterized in that it is derived from a group B type 35 adenovirus (Ad5) fiber knob.

10. The Ad35 adenovirus fiber according to claim 9, characterized in that it is modified by mutation of a residue in the AB, EF or HI exposed loop.

11. The adenovirus fiber according to claim 6, characterized in that it is derived and Ad2 fiber.

12. The adenovirus fiber according to claim 11, characterized in that it is modified by mutation of a residue in the AB, EF or HI exposed loop.

13. The adenovirus fiber according to claim 1, wherein the binding affinity of the fiber for its native cellular receptor is not substantially reduced.

14. The adenovirus fiber of claim 1, wherein binding to the blood factor protein is ablated.

15. The adenovirus fiber according to claim 1, comprising a ligand capable of recognizing a cell surface molecule different from the native cellular receptor for adenovirus fiber.

16. The adenovirus fiber according to claim 15, characterized in that the ligand is an antibody, a peptide, a lipid, a glycolipid, or a sugar.

17. The adenovirus fiber according to claim 15, characterized in that the ligand is inserted at the C-terminal end of the fiber, in the HI loop, in capsid protein IX, in the penton or in the hexon.

18. A DNA fragment or expression vector encoding the adenovirus fiber according to claim 1.

19. A cell line comprising the DNA fragment of claim 18 in a form integrated into the genome or in the form of an episome, wherein the DNA fragment is operatively linked to a promoter for expression of the adenovirus fiber in the cell line.

20. The cell line according to claim 19, further comprising a function encoded by the E1, E2, E4 or L1-L5 region and capable of complementing an adenovirus deficient in a function encoded by the E1, E2, E4 or L1-L5 region.

21. The cell line according to claim 19, characterized in that it is derived from a 293 cell line.

22. An adenovirus comprising the adenovirus fiber according to claim 1.

23. An adenovirus characterized in that it lacks a functional native fiber and in that it comprises a fiber according to claim 1 and a ligand capable of recognizing a cell surface molecule different from the native cellular receptor for the adenovirus.

24. The adenovirus according to claim 23, characterized in that the ligand is an antibody, a peptide, a lipid, a polypeptide, a glycolipid or a sugar.

25. The adenovirus according to claim 23, characterized in that the ligand is inserted at the C-terminal end of the fiber, in the HI loop, in capsid protein IX, in the penton or in the hexon.

26. The adenovirus according to claim 23, characterized in that the ligand is inserted into an adenovirus capsid protein.

27. The adenovirus according to claim 26, characterized in that the ligand is inserted into the hexon, the penton or the fiber.

28. The adenovirus according to claim 22, characterized in that it is a replication-defective or replication-attenuated recombinant adenovirus.

29. The adenovirus according to claim 28, characterized in that it is deleted for all or part of the E1 region and, optionally, for all or part of the E3 region.

30. The adenovirus according to claim 29, characterized in that it is further deleted for all or part of the E2, E4 or L1-L5 region.

31. The adenovirus according to claim 29, characterized in that it comprises a gene of interest selected from a cytokine, a cellular receptor, a nuclear receptor, a ligand, a coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection, an antibody, a toxin, an immunotoxin or a marker.

32. A method of producing an adenovirus according to claim 22, comprising:
transfecting a genome encoding the adenovirus into a host cell line,
culturing the transfected host cell line under appropriate conditions to allow the production of the adenovirus,
recovering the adenovirus from the culture of the transfected host cell line, and
optionally substantially purifying the adenovirus.

33. A host cell infected with an adenovirus according to claim 22.

34. A pharmaceutical composition comprising an adenovirus according to claim 22 in combination with a pharmaceutically acceptable carrier.

35. The pharmaceutical of claim 34, wherein the adenovirus is a replication-defective or replication-attenuated recombinant adenovirus.

36. The adenovirus of claim 35, characterized in that it comprises a gene of interest selected from a cytokine, a cellular receptor, a nuclear receptor, a ligand, a coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection,. an antibody, a toxin, an immunotoxin or a marker.

37. A pharmaceutical composition comprising an infected host cell according to claim 33 in combination with a pharmaceutically acceptable carrier.

38. The pharmaceutical of claim 37, wherein the adenovirus is a replication-defective or replication-attenuated recombinant adenovirus.

39. The pharmaceutical of claim 38, characterized in that it comprises a gene of interest selected from a cytokine, a cellular receptor, a nuclear receptor, a ligand, a coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection, an antibody, a toxin, an immunotoxin or a marker.

40. The therapeutic or prophylactic use of an adenovirus according to claim 22, for the preparation of a medicament intended for the treatment of the human or animal body by gene therapy.

41. A method for increasing the efficacy of adenoviral vector administration, comprising:
administering to a subject a recombinant adenovirus vector comprising a gene of interest and encoding a mutant adenoviral fiber comprising a binding site for a cellular receptor and a mutated blood factor protein binding site, wherein the affinity of the fiber for the blood factor protein is substantially reduced.

42. The method according to claim 41, wherein the cellular receptor is a native cellular receptor.

43. The method according to claim 41, wherein the binding site for the cellular receptor is a ligand.

44. The method according to claim 43, wherein the ligand is an antibody, a peptide, a hormone, a polypeptide or a sugar.

45. The method according to claim 41, wherein the gene of interest is a cytokine, a cellular receptor, a nuclear receptor, a ligand, a coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection, an antibody, a toxin, an immunotoxin or a marker.

46. The method according to claim 41, wherein the gene of interest is operatively linked to a promoter.

47. The method according to claim 46, wherein the promoter is a tissue-specific promoter.

48. A method for reducing toxicity associated with adenovirus administration, comprising:
administering to a subject a recombinant adenovirus or adenoviral vector comprising a gene of interest and a mutant adenoviral fiber comprising a binding site for a cellular receptor and a mutated blood factor protein binding site, wherein the affinity of the fiber for the blood factor protein is substantially reduced.

49. The method according to claim 48, wherein the cellular receptor is a native cellular receptor.

50. The method according to claim 48, wherein the binding site for the cellular receptor is a ligand.

51. The method according to claim 50, wherein the ligand is an antibody, a peptide, a hormone, a polypeptide or a sugar.

52. The method according to 48, wherein the gene of interest is a cytokine, a cellular receptor, a nuclear receptor, a ligand, a coagulation factor, a CFTR protein, insulin, dystrophin, a growth hormone, an enzyme, an enzyme inhibitor, a polypeptide with antitumor effect, a polypeptide capable of inhibiting a bacterial, parasitic or viral infection, an antibody, a toxin, an immunotoxin or a marker.


SEQUENCE LISTING (8 sequences)

SEQ ID NO: 1 - protein, 185 aa, adenovirus
DKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 2 - protein, 185 aa, adenovirus
DKLTLWTTPDPSPNCRIHSDNDCKFTLVLTKCGSQVLATVAALAVSGDLSSMTGTVPSVSIFLRFDQNGVLMENSSLKKHYWNFRNGNSTNANPYTNAVGFMPNLLAYPKTQSQTKNNIVSQVYLHGDKTKPMILTITLNGTSESTETSEVSTYSMSFTWSWESGKYTTETFATNSYTFSYIAQE

SEQ ID NO: 3 - protein, 182 aa, adenovirus
DPLTLWTTPDPPPNCSLIQELDAKLTLCLTKNGSIVNGIVSLVGVKGNLLNIQSTTTTVGVHLVFDEQGRLITSTPTALVPQASWGYRQGQSVSTNTVTNGLGFMPNVSAYPRPNASEAKSQMVSLTYLQGDTSKPITMKVAFNGITSLNGYSLTFMWSGLSNYINQPFSTPSCSFSYITQE

SEQ ID NO: 4 - protein, 182 aa, adenovirus
DPLTLWTTPDPPPNCTLRQELDAKLTLCLTKNESIVNGIVSLIGVKGDLLHIQPTTTTVGLHLVFDRQGRLVTTTPTALVPQASWGYKQGQSVSSSAVANALGFMPNVSAYPRPNAGEAKSQMLSQTYLQGDTTKPITMKVVFNGNATVDGYSLTFMWTGVSNYLNQQFSTPSCSFSYIAQE

SEQ ID NO: 5 - protein, 185 aa, adenovirus
DKRTLWTTPDTSPNCKIDQDKDSKLTLVLTKCGSQILANVSLIVVDGKYKIINNNTQPALKGFTIKLLFDENGVLMESSNLGKSYWNFRNENSIMSTAYEKAIGFMPNLVAYPKPTAGSKKYARDIVYGNIYLGGKPDQPVTIKTTFNQETGCEYSITFDFSWAKTYVNVEFETTSFTFSYIAEQ

SEQ ID NO: 6 - protein, 194 aa, adenovirus
SINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 7 - protein, 196 aa, adenovirus
NINTLWTGINPTEANCQMMDSSESNDCKLILTLVKTGALVTAFVYVIGVSNNFNMLTTYRNINFTAELFFDSAGNLLTSLSSLKTPLNHKSGQNMATGAITNAKSFMPSTTAYPFNNNSREKENYIYGTCHYTASDHTAFPIDISVMLNQRAIRADTSYCIRITWSWNTGDAPEGQTSATTLVTSPFTFYYIREDD

SEQ ID NO: 8 - protein, 12 aa, adenovirus
SKCDCRGECFCD